(19) United States
(12) Patent Application Publication
(10) Pub. No.: US 2025/0263758 A1
(43) Pub. Date: Aug. 21, 2025

(54) A CELL-FREE BIO-MANUFACTURING PLATFORM FOR PRODUCTION OF FATTY ACIDS AND CANNABINOIDS

(71) Applicant: Northwestern University, Evanston, IL (US)

(72) Inventors: Michael C. Jewett, Evanston, IL (US); Bastian Vogeli, Evanston, IL (US); Luca Schulz, Evanston, IL (US)

(21) Appl. No.: 18/251,604

(22) PCT Filed: Nov. 2, 2021
(86) PCT No.: PCT/US21/72188

§ 371 Date: May 3, 2023

Related U.S. Application Data
(60) Provisional application No. 63/109,686, filed on Nov. 4, 2020.

(51) Int. Cl. C12P 7/42; C12N 9/10; C12N 9/16; C12N 9/88; C12P 7/40

(52) U.S. Cl. CPC C12P 7/42; C12N 9/1085; C12N 9/16; C12N 9/88; C12P 7/40; C12Y 404/01026

(57) ABSTRACT

The invention relates to cell-free systems, methods, and kits for bio-manufacturing natural or chemical products from readily available feedstocks, such as glucose. The systems, methods, and kits allow for cell-free bio-manufacturing of desired products in cell-free conditions, and the rapid optimization of conditions for preparing the products in cell-free conditions. Disclosed herein are systems, methods, and kits for the cell-free production of fatty acids, cannabinoids, and their intermediates.

### CROSS-REFERENCE TO RELATED APPLICATIONS

This application claims the benefit of U.S. Provisional Application No. 63/109,686, with filing date Nov. 4, 2020, the content of which is incorporated by reference herein in its entirety.

### STATEMENT REGARDING FEDERALLY SPONSORED RESEARCH OR DEVELOPMENT

This invention was made with government support under EE0008354 awarded by the U.S. Department of Energy. The government has certain rights in the invention.

### BACKGROUND

The invention relates to cell-free systems, methods, and kits for bio-manufacturing a desired product from readily available feedstocks, such as glucose. The systems, methods, and kits allow for cell-free bio-manufacturing of chemical products or natural products in cell-free conditions, and the rapid optimization of conditions for preparing a chemical or natural products in cell-free conditions. Particularly disclosed herein are systems, methods, and kits for the cell-free production of fatty acids, cannabinoids, and their intermediates.

Cannabinoids form a group of more than 100 compounds that interact with the human endocannabinoid system and are therefore promising pharmacological agents. They are produced by Cannabis sativa from fatty acid and isoprenoid precursors. Isolation of even highly produced cannabinoids is challenging due to the high similarity with other cannabinoids. Chemical synthesis is complex and often leads to low yields. Implementation into microorganisms has led to low product yields, due to limiting supply of the fatty acid derivative hexanoyl-CoA, as well as toxicity of multiple pathway intermediates.

Here, we present an in vitro bio-manufacturing platform for the synthesis of olivetolic acid and cannabigerolic acid (CBGA) from glucose alone. The combination of an optimized reverse ß-oxidation pathway for the synthesis of hexanoyl-CoA (1.5 g/L) with biosynthetic enzymes from C. sativa yielded 50 mg/L olivetolic acid, which is 5× higher than comparable engineered E. coli strains and 15× higher than engineered yeast. We then show that olivetolic acid can be coupled with a previously developed GPP-producing in vitro platform and the engineered prenyltransferase NphB7 to produce the cannabinoid precursor CBGA. This forms the starting point of a platform able to synthesize cannabinoids and cannabinoid derivatives completely in vitro from simple carbon feedstocks.

### BRIEF DESCRIPTION OF THE FIGURES

FIG. 1A-D. Characterization of extract background strains BL21*, MB263, JC01 and JST07. (A) Optimized GFP yields. (B) Side product formation. (C) TE background activity. (D) ¹³C-glucose incorporation into hexanoic acid.

FIG. 2A-D. (A) Initial combinatorial screen to find a starting enzyme combination for down scaling to 96-well plate assays. (B) 96-well plate based screen to optimize cofactor and buffer conditions for the base case r-BOX enzymes. pH, % of fresh extract, coenzyme A and NAD⁺ were tested combinatorially resulting in 81 conditions measured in triplicates. The fifth best condition overall (2332) was picked for the following enzymatic screen. (C) Representative phylogenetic tree of hydroxybutyrate dehydratases (all other trees in FIG. 9A-D). A diverse set of enzyme homologues were picked for gene synthesis, CFPS and CFME. (D) Yield of soluble r-BOX enzymes as determined by ¹⁴C-leucine incorporation using CFPS in JST07 extract. Homologues of all enzyme classes expressed above our cut off of 1 mM.

FIG. 3A-B. Combinatorial screen of r-BOX enzyme homologues. (A) Overview of r-BOX and the enzymes used as the base case. Termination enzymes were not included in the analysis and Ecol_tesA was kept constant at 150 nM in all assays. The base case was adapted based on the stereospecificity of involved enzymes as necessary. (B) Hexanoic acid (2 iterative elongations) production by the investigated enzyme combinations. Full enzyme names, abbreviations and organism or origin listed in Table 1. Top left, TL×HBD; top right, HBD×CRT; bottom left, CRT×TER; bottom right, TER×TL. All rBOX enzymes at 300 nM with 150 nM thioesterase. Incubation at 30° C. for 24 h. n=3, error bars=not shown.

FIG. 4A-C. Acetyl-CoA carboxylase enriched extracts enable the production of olivetolic acid and derivatives. (A) Scheme of the added biosynthetic module. Starting from glucose, olivetolic acid is synthesized by iterative extension of hexanoyl-CoA with malonyl-CoA, catalyzed by a Type III PKS (TKS). (B) SDS-PAGE analysis confirms the in vivo overexpression of accBCDE. The produced proteins and their calculated molecular mass are labeled. Middle lane shows a blank E. coli JST07 extract. (C) Production of olivetolic acid and side products is only observed in the presence of accBCDA enriched extract (here 4% v/v accBCDA extract in assay). OA producing reactions contained 300 nM of each r-BOX enzyme, TKS and OAC. Addition of 50 mM NaHCO₃ increases final product titers. m/z: 223.1 for olivetolic acid, 251.1 for hepty-chain derivative. Negative polarization. 24 h, 30° C. n=3, error bars=SD.

FIG. 5A-B. Small-scale optimization of olivetolic acid production more than doubles product titers and improves the product to side product ratio. (A) Adding excess type III PKS enzymes, relative to rBOX enzymes, increases OA titers. An optimum of accBCDA-enriched extract is found at 2% v/v and product titers increase linearly with increasing bicarbonate concentrations. (B) Adding excess type III PKS enzymes does not reduce the produced amount of side product but leads to higher OA titers, improving the relative ratio between the two products. Data is taken from A. 24 h, 30° C.; n=3, error bars=SD.

FIG. 6A-C. Cerulenin, an antibiotic that inhibits fatty acid biosynthesis, increases OA titers fivefold. (A) Structure of Cerulenin. (B) Scheme of olivetolic acid biosynthesis with the inhibiting action of cerulenin highlighted (red). (C) Effect of cerulenin in increasing concentrations on the production of olivetolic acid. No heptyl-chain derivative is observed. Assays contain 600 nM TKS and OAC, 200 nM each rBOX enzyme (optimized combination), no TE. 24 h, 30° C. n=3, error bars=SD.

FIG. 7A-B. Conversion of olivetolic acid to cannabigetolic acid in vitro. (A) Reaction scheme of the investigated branch of the cannabinoid biosynthetic route. (B) LC signal of the completed OA to CBGA conversion using CFPS produced enzymes in 15 mL and 100 mL reactions. Reactions contained 10% HMGS/R & AtoB-enriched lysate, 300 nM MK, PMK, PMD; 600 nM IDI; 800 nM GPPS and 600 nM NphB7. Additionally, 1 mM ATP was added exogenously. Incubation at 30° C. for 7 h.

FIG. 8. Exemplary standard curve of various concentrations of hexanoic acid versus extracted ion counts.

FIG. 9A-D. Phylogenetic trees of (A) TL, thiolase (B) CRT, Short-chain-enoyl-CoA hydratase (C) TER, Trans-2-enoyl-CoA reductase (D) TE, thioesterase.

### SUMMARY

Disclosed herein are systems, methods, and kits for the cell-free production of fatty acids, cannabinoids, and their intermediates. In some embodiments, systems, methods and kits are provided for the cell-free production of hexanoic acid, olivetolic acid, cannabigerolic acid, or intermediates thereof from readily available feedstock such as glucose or the products of glycolysis.

In one aspect of the current disclosure, methods for the enzymatic preparation of hexanoic acid or an intermediate of hexanoic acid in a hexanoic acid synthetic pathway in vitro from a feedstock comprising glucose or a product of glycolysis that reacts with one or more enzymes to produce the hexanoic acid or the intermediate of hexanoic acid in the hexanoic acid synthetic pathway are provided. In some embodiments, the method comprises: (a) reacting a cell-free protein synthesis reaction mixture, the cell-free protein synthesis reaction mixture comprising a cellular extract from a host strain, a translation template encoding the one or more enzymes, and cell-free protein synthesis reagents, (b) expressing the translation template in the cell-free protein synthesis reaction mixture to prepare the one or more enzymes, (c) combining the cell-free protein synthesis reaction mixture and the feedstock to form a secondary reaction mixture, wherein the feedstock reacts in the presence of the one or more enzymes to produce the hexanoic acid or the intermediate of hexanoic acid in the hexanoic acid synthetic pathway; wherein the one or more enzymes are selected from the group consisting of acetyl-CoA acetyltransferase (ThlA), β-hydroxybutyryl-CoA dehydrogenase (Hbd1), 3-hydroxybutyryl-CoA dehydratase (Crt), trans-enoyl-CoA reductase (Ter), thioesterase 1 (TesA), and combinations thereof; and wherein the intermediate of hexanoic acid in the hexanoic acid synthetic pathway is selected from acetyl-CoA, acetoacetyl-CoA, 3-hydroxybutyryl-CoA, hex-(2E)-enoyl-CoA, and hexanoyl-CoA. In some embodiments, the methods comprise adding NAD+ and coenzyme A to the secondary reaction mixture. In some embodiments, the host strain for the cellular extract comprises Escherichia coli (E. coli). In some embodiments, the host strain comprises one or more of E. coli strain BL21, JS07, MB263, MB263sucD and JC01. In some embodiments, the host strain comprises JS07. In some embodiments, the cell-free protein synthesis reaction mixture and the secondary reaction mixture are in separate reaction vessels. In some embodiments, the cell-free protein synthesis reaction and the secondary reaction are in the same reaction vessel.

In another aspect of the current disclosure, methods for the enzymatic preparation of olivetolic acid or an intermediate of olivetolic acid in a olivetolic acid synthetic pathway in vitro from a feedstock comprising glucose or a product of glycolysis and hexanoyl-CoA (optionally prepared by the method of claim 1) that reacts with one or more enzymes to produce the olivetolic acid or the intermediate of olivetolic acid in the olivetolic acid synthetic pathway are provided. In some embodiments, the methods comprise: (a) reacting a cell-free protein synthesis reaction mixture, the cell-free protein synthesis reaction mixture comprising a cellular extract from a host strain, a translation template encoding the one or more enzymes, and cell-free protein synthesis reagents, (b) expressing the translation template in the cell-free protein synthesis reaction mixture to prepare the one or more enzymes, (c) combining the cell-free protein synthesis reaction mixture and the feedstock to form a secondary reaction mixture, wherein the feedstock reacts in the presence of the one or more enzymes to produce the olivetolic acid or the intermediate of olivetolic acid in the olivetolic acid synthetic pathway; wherein the one or more enzymes are selected from the group consisting of acetyl coenzyme A carboxylase (AccBCDA) and biotin-[acetyl-CoA-carboxylase]ligase (BirA), 3,5,7-Trioxododecanoyl-CoA synthase (TKS), olivetolic acid cyclase (OAC), and combinations thereof; and wherein the intermediate of olivetolic acid in the olivetolic acid synthetic pathway is selected from acetyl-CoA, malonyl-CoA, hexanoyl-CoA, and 3,5,7-trioxododecanoyl-CoA. In some embodiments, the methods comprise adding cerulenin to the secondary reaction mixture. In some embodiments, the host strain for the cellular extract comprises Escherichia coli (E. coli). In some embodiments, the host strain comprises one or more of E. coli strain BL21, JS07, MB263, MB263sucD and JC01. In some embodiments, the host strain comprises JS07. In some embodiments, the cell-free protein synthesis reaction mixture and the secondary reaction mixture are in separate reaction vessels. In some embodiments, the cell-free protein synthesis reaction and the secondary reaction are in the same reaction vessel.

In another aspect of the current disclosure, methods for the enzymatic preparation of cannabigerolic acid or an intermediate of cannabigerolic acid in a cannabigerolic acid synthetic pathway in vitro from a feedstock comprising glucose or a product of glycolysis and olivetolic acid (optionally prepared by the method of claim 2) that reacts with one or more enzymes to produce the cannabigerolic acid or the intermediate of cannabigerolic acid in the cannabigerolic acid synthetic pathway are provided. In some embodiments, the methods comprise: (a) reacting a cell-free protein synthesis reaction mixture, the cell-free protein synthesis reaction mixture comprising a cellular extract from a host strain, a translation template encoding the one or more enzymes, and cell-free protein synthesis reagents, (b) expressing the translation template in the cell-free protein synthesis reaction mixture to prepare the one or more enzymes, (c) combining the cell-free protein synthesis reaction mixture comprising the feedstock to form a secondary reaction mixture, wherein the feedstock reacts in the presence of the one or more enzymes to produce the cannabigerolic acid or the intermediate of cannabigerolic acid in the cannabigerolic acid synthetic pathway; wherein the one or more enzymes are selected from the group consisting of acetyl-CoA acetyltransferase (AtoB), 3-hydroxy-3-methyl-glutaryl-CoA synthase (HMGS), 3-hydroxy-3-methyl-glutaryl-coenzyme A reductase (HMGR), mevalonate kinase (Mk), phosphomevalonate kinase (PMK), pyrophosphomevalonate decarboxylase (PMD), isopentenyl pyrophosphate isomerase (IDI), geranyl diphosphate synthase (GPPS), and prenyltransferase NphB7, and combinations thereof; and wherein the intermediate of cannabigerolic acid in the cannabigerolic acid synthetic pathway is selected from acetyl-CoA, mevalonate, and geranyl pyrophosphate. In some embodiments, the host strain for the cellular extract comprises Escherichia coli (E. coli). In some embodiments, the host strain comprises one or more of E. coli strain BL21, JS07, MB263, MB263sucD and JC01. In some embodiments, the host strain comprises JS07.

In another aspect of the current disclosure, kits are provided. In some embodiments, the kits comprise: a) a first composition comprising acetyl-CoA acetyltransferase (ThlA), β-hydroxybutyryl-CoA dehydrogenase (Hbd1), 3-hydroxybutyryl-CoA dehydratase (Crt), trans-enoyl-CoA reductase (Ter), thioesterase 1 (TesA); b) a second composition comprising acetyl coenzyme A carboxylase (AccBCDA) and biotin-[acetyl-CoA-carboxylase]ligase (BirA), 3,5,7-Trioxododecanoyl-CoA synthase (TKS), olivetolic acid cyclase (OAC); and c) a third composition comprising acetyl-CoA acetyltransferase (AtoB), 3-hydroxy-3-methyl-glutaryl-CoA synthase (HMGS), 3-hydroxy-3-methyl-glutaryl-coenzyme A reductase (HMGR), mevalonate kinase (Mk), phosphomevalonate kinase (PMK), pyrophosphomevalonate decarboxylase (PMD), isopentenyl pyrophosphate isomerase (IDI), geranyl diphosphate synthase (GPPS), and prenyltransferase NphB7. In some embodiments, the second composition comprises cerulenin.

In some embodiments, at least one of the first, second, and third composition comprises a cell extract. In some embodiments, the cell extract comprises an E. coli cell extract. In some embodiments, the E. coli extract comprises a JS07 extract.

### DETAILED DESCRIPTION

Disclosed herein are systems, methods, and kits for the cell-free production of fatty acids, cannabinoids, and their intermediates. In some embodiments, the methods involve one or more cell-free protein synthesis (CFPS) reactions, followed by one or more reactions including the CFPS product(s) and one or more feedstocks to produce the desired product or intermediate.

In one aspect an in vitro bio-manufacturing method is provided to produce a desired product. In one embodiment, the method comprises providing a cell-free protein synthesis (CFPS) reaction mixture to a protein reaction vessel, expressing a translation template in the protein reaction vessel to prepare an enzyme, and providing the enzyme to a reaction mixture comprising a feedstock to a secondary reaction vessel, wherein the feedstock reacts in the presence of the enzyme to produce the desired product. In certain embodiments, the protein reaction vessel and the secondary reaction vessel are the same vessel. Methods for cell-free protein synthesis are provided in WO/2014/144583 to Jewett et al., the disclosure of which is incorporated by reference in its entirety.

In certain embodiments, the method may include expressing one or more additional enzymes in a second protein reaction vessel. The second protein reaction vessel may be the same vessel as the protein reaction vessel.

As used herein, a “reaction vessel” or “protein reaction vessel” refers to a suitable vessel for performing the indicated reaction. In some embodiments, the reaction vessel is a flask, beaker, reactor, or the like.

In certain embodiments, the method may further comprise providing a transcription template to prepare the translation template. Where the transcription template is provided, one may additionally provide a polymerase and nucleoside triphosphates (NTPs): ATP, GTP, CTP, and UTP. Preparation of the translation template and preparation for the enzyme from the translation template may occur in the same reaction vessel or different reaction vessels.

In some embodiments, the cellular extract may provide natural enzyme metabolism from the host strain that may be exploited to perform desired chemical modifications. As used herein, “host strain” refers to the strain of microorganism used to express the products that comprise the cellular extract. Natural enzyme metabolism means any process or chemical, including cellular extract enzymes, which may be necessary or beneficial for desired molecular transformations. Natural enzyme metabolism may provide energy, which may facilitate the desired molecular transformations. Natural enzyme metabolism may provide cofactor regeneration, which may facilitate the desired molecular transformation. Natural enzyme metabolism may also provide cellular extract enzymes. In certain embodiments, the cellular extract enzyme may be one or more heterologous enzymes expressed by the host. In certain embodiments, the cellular extract enzyme may be one or more native enzyme expressed by the host. In certain embodiments, the cellular extract enzyme may be a combination of one of more heterologous enzymes and native enzymes expressed by the host. In certain embodiments, the cellular extract enzyme is overexpressed by the host to enrich the extract with the cellular extract enzyme. In certain embodiments, the cellular extract enzyme may transform a chemical product or natural product into a feedstock. In other embodiments, the cellular extract enzyme may further transform the chemical product or the natural product that is formed by the reaction of the feedstock in the presence of the enzyme expressed by cell-free protein synthesis.

The method further includes providing the enzyme and a feedstock to a secondary reaction vessel. The protein synthesis reaction vessel and the secondary reaction vessel may be the same reaction vessel.

In some embodiments, a method for the enzymatic preparation of a chemical product or natural product in vitro, includes providing a cell-free protein synthesis reaction mixture to a protein reaction vessel, the cell-free protein synthesis reaction mixture comprising a cellular extract from a host strain, a translation template, and cell-free protein synthesis reagents, expressing the translation template in the protein reaction vessel to prepare an enzyme, providing the enzyme, the cellular extract, and a secondary reaction mixture to a secondary reaction vessel, the secondary reaction mixture comprising a feedstock, wherein the feedstock reacts in the presence of the enzyme to prepare the chemical product or the natural product and wherein the cellular extract provides, if necessary, natural enzyme metabolism from the host strain. In some embodiments, the objective of the method is to provide hexanoic acid, olivetolic acid, cannabigerolic acid, or intermediates thereof.

### Kits

In one aspect of the invention, kits for cell-free bio-manufacturing according to the methods of the present invention are disclosed. Kits for cell-free bio-manufacturing comprising one or more components for the practice of the cell-free biomanufacturing methods. In one aspect, the kits may comprise one or more components, individually or collectively, for the practice of CFPS. In certain embodiments, the kit may comprise a CFPS reaction mixture or the individual solutes or solutions that may be combined to form a CFPS reaction mixture. In one aspect, the kits may comprise one or more components, individually or collectively, for the practice of the secondary reaction. In certain embodiments, the kit may comprise a secondary reaction mixture or the individual solutes or solutions that may be combined to form a secondary reaction mixture. In certain embodiments, the kit further comprises a feedstock.

### Definitions

To aid in understanding the invention, several terms are defined below.

Unless defined otherwise, all technical and scientific terms used herein have the same meaning as commonly understood by one of skill in the art. Although any methods and materials similar to or equivalent to those described herein can be used in the practice or testing of the claims, the exemplary methods and materials are described herein.

Moreover, reference to an element by the indefinite article “a” or “an” does not exclude the possibility that more than one element is present, unless the context clearly requires that there be one and only one element. The indefinite article “a” or “an” thus usually means “at least one.”

The term “about” means within a statistically meaningful range of a value or values such as a stated concentration, length, molecular weight, pH, time frame, temperature, pressure or volume. Such a value or range can be within an order of magnitude, typically within 20%, more typically within 10%, and even more typically within 5% of a given value or range. The allowable variation encompassed by “about” will depend upon the particular system under study.

The terms “comprising,” “having,” “including,” and “containing” are to be construed as open-ended terms (i.e., meaning “including, but not limited to,”) unless otherwise noted.

Recitation of ranges of values herein are merely intended to serve as a shorthand method of referring individually to each separate value falling within the range, and includes the endpoint boundaries defining the range, unless otherwise indicated herein, and each separate value is incorporated into the specification as if it were individually recited herein.

The terms “nucleic acid” and “oligonucleotide,” as used herein, refer to polydeoxyribonucleotides (containing 2-deoxy-D-ribose), polyribonucleotides (containing D-ribose), and to any other type of polynucleotide that is an N glycoside of a purine or pyrimidine base. There is no intended distinction in length between the terms “nucleic acid”, “oligonucleotide” and “polynucleotide”, and these terms will be used interchangeably. These terms refer only to the primary structure of the molecule. Thus, these terms include double- and single-stranded DNA, as well as double- and single-stranded RNA. For use in the present invention, an oligonucleotide also can comprise nucleotide analogs in which the base, sugar or phosphate backbone is modified as well as non-purine or non-pyrimidine nucleotide analogs.

Oligonucleotides can be prepared by any suitable method, including direct chemical synthesis by a method such as the phosphotriester method of Narang et al., 1979, Meth. Enzymol. 68:90-99; the phosphodiester method of Brown et al., 1979, Meth. Enzymol. 68:109-151; the diethylphosphoramidite method of Beaucage et al., 1981, Tetrahedron Letters 22:1859-1862; and the solid support method of U.S. Pat. No. 4,458,066, each incorporated herein by reference. A review of synthesis methods of conjugates of oligonucleotides and modified nucleotides is provided in Goodchild, 1990, Bioconjugate Chemistry 1(3): 165-187, incorporated herein by reference.

The term “primer,” as used herein, refers to an oligonucleotide capable of acting as a point of initiation of DNA synthesis under suitable conditions. Such conditions include those in which synthesis of a primer extension product complementary to a nucleic acid strand is induced in the presence of four different nucleoside triphosphates and an agent for extension (for example, a DNA polymerase or reverse transcriptase) in an appropriate buffer and at a suitable temperature.

A primer is preferably a single-stranded DNA. The appropriate length of a primer depends on the intended use of the primer but typically ranges from about 6 to about 225 nucleotides, including intermediate ranges, such as from 15 to 35 nucleotides, from 18 to 75 nucleotides and from 25 to 150 nucleotides. Short primer molecules generally require cooler temperatures to form sufficiently stable hybrid complexes with the template. A primer need not reflect the exact sequence of the template nucleic acid, but must be sufficiently complementary to hybridize with the template. The design of suitable primers for the amplification of a given target sequence is well known in the art and described in the literature cited herein.

Primers can incorporate additional features which allow for the detection or immobilization of the primer but do not alter the basic property of the primer, that of acting as a point of initiation of DNA synthesis. For example, primers may contain an additional nucleic acid sequence at the 5′ end which does not hybridize to the target nucleic acid, but which facilitates cloning or detection of the amplified product, or which enables transcription of RNA (for example, by inclusion of a promoter) or translation of protein (for example, by inclusion of a 5′-UTR, such as an Internal Ribosome Entry Site (IRES) or a 3′-UTR element, such as a poly(A)n sequence, where n is in the range from about 20 to about 200). The region of the primer that is sufficiently complementary to the template to hybridize is referred to herein as the hybridizing region.

The term “promoter” refers to a cis-acting DNA sequence that directs RNA polymerase and other trans-acting transcription factors to initiate RNA transcription from the DNA template that includes the cis-acting DNA sequence.

The terms “target, “target sequence”, “target region”, and “target nucleic acid,” as used herein, are synonymous and refer to a region or sequence of a nucleic acid which is to be amplified, sequenced or detected.

The term “hybridization,” as used herein, refers to the formation of a duplex structure by two single-stranded nucleic acids due to complementary base pairing. Hybridization can occur between fully complementary nucleic acid strands or between “substantially complementary” nucleic acid strands that contain minor regions of mismatch. Conditions under which hybridization of fully complementary nucleic acid strands is strongly preferred are referred to as “stringent hybridization conditions” or “sequence-specific hybridization conditions”. Stable duplexes of substantially complementary sequences can be achieved under less stringent hybridization conditions; the degree of mismatch tolerated can be controlled by suitable adjustment of the hybridization conditions. Those skilled in the art of nucleic acid technology can determine duplex stability empirically considering a number of variables including, for example, the length and base pair composition of the oligonucleotides, ionic strength, and incidence of mismatched base pairs, following the guidance provided by the art (see, e.g., Sambrook et al., 1989, Molecular Cloning-A Laboratory Manual, Cold Spring Harbor Laboratory, Cold Spring Harbor, New York; Wetmur, 1991, Critical Review in Biochem. and Mol. Biol. 26(3/4):227-259; and Owczarzy et al., 2008, Biochemistry, 47: 5336-5353, which are incorporated herein by reference).

The term “amplification reaction” refers to any chemical reaction, including an enzymatic reaction, which results in increased copies of a template nucleic acid sequence or results in transcription of a template nucleic acid. Amplification reactions include reverse transcription, the polymerase chain reaction (PCR), including Real Time PCR (see U.S. Pat. Nos. 4,683,195 and 4,683,202; PCR Protocols: A Guide to Methods and Applications (Innis et al., eds, 1990)), and the ligase chain reaction (LCR) (see Barany et al., U.S. Pat. No. 5,494,810). Exemplary “amplification reactions conditions” or “amplification conditions” typically comprise either two or three step cycles. Two-step cycles have a high temperature denaturation step followed by a hybridization/elongation (or ligation) step. Three step cycles comprise a denaturation step followed by a hybridization step followed by a separate elongation step.

As used herein, a “polymerase” refers to an enzyme that catalyzes the polymerization of nucleotides. “DNA polymerase” catalyzes the polymerization of deoxyribonucleotides. Known DNA polymerases include, for example, Pyrococcus furiosus (Pfu) DNA polymerase, E. coli DNA polymerase I, T7 DNA polymerase and Thermus aquaticus (Taq) DNA polymerase, among others. “RNA polymerase” catalyzes the polymerization of ribonucleotides. The foregoing examples of DNA polymerases are also known as DNA-dependent DNA polymerases. RNA-dependent DNA polymerases also fall within the scope of DNA polymerases. Reverse transcriptase, which includes viral polymerases encoded by retroviruses, is an example of an RNA-dependent DNA polymerase. Known examples of RNA polymerase (“RNAP”) include, for example, T3 RNA polymerase, T7 RNA polymerase, SP6 RNA polymerase and E. coli RNA polymerase, among others. The foregoing examples of RNA polymerases are also known as DNA-dependent RNA polymerase. The polymerase activity of any of the above enzymes can be determined by means well known in the art.

As used herein, a primer is “specific,” for a target sequence if, when used in an amplification reaction under sufficiently stringent conditions, the primer hybridizes primarily to the target nucleic acid. Typically, a primer is specific for a target sequence if the primer-target duplex stability is greater than the stability of a duplex formed between the primer and any other sequence found in the sample. One of skill in the art will recognize that various factors, such as salt conditions as well as base composition of the primer and the location of the mismatches, will affect the specificity of the primer, and that routine experimental confirmation of the primer specificity will be needed in many cases. Hybridization conditions can be chosen under which the primer can form stable duplexes only with a target sequence. Thus, the use of target-specific primers under suitably stringent amplification conditions enables the selective amplification of those target sequences that contain the target primer binding sites.

As used herein, “expression template” refers to a nucleic acid that serves as substrate for transcribing at least one RNA that can be translated into a polypeptide or protein. Expression templates include nucleic acids composed of DNA or RNA. Suitable sources of DNA for use a nucleic acid for an expression template include genomic DNA, cDNA and RNA that can be converted into cDNA. Genomic DNA, cDNA and RNA can be from any biological source, such as a tissue sample, a biopsy, a swab, sputum, a blood sample, a fecal sample, a urine sample, a scraping, among others. The genomic DNA, cDNA and RNA can be from host cell or virus origins and from any species, including extant and extinct organisms. As used herein, “expression template” and “transcription template” have the same meaning and are used interchangeably.

As used herein, “translation template” refers to an RNA product of transcription from an expression template that can be used by ribosomes to synthesize polypeptides or proteins.

The term “reaction mixture,” as used herein, refers to a solution containing reagents necessary to carry out a given reaction. A reaction mixture is referred to as complete if it contains all reagents necessary to enable the reaction, and incomplete if it contains only a subset of the necessary reagents.

An “amplification reaction mixture”, which refers to a solution containing reagents necessary to carry out an amplification reaction, typically contains oligonucleotide primers and a DNA polymerase in a suitable buffer.

A “PCR reaction mixture”, which refers to a solution containing the reagents necessary to carry out a PCR reaction, typically contains DNA polymerase, dNTPs, and a divalent metal cation in a suitable buffer.

A “cell-free protein synthesis (CFPS) reaction mixture”, which refers to a solution containing the reagents necessary to carry out CFPS, typically contains a crude or partially-purified bacterial or yeast extract, an RNA translation template, and a suitable reaction buffer for promoting cell-free protein synthesis from the RNA translation template. In some aspects, the CFPS reaction mixture can include exogenous RNA translation template. In other aspects, the CFPS reaction mixture can include a DNA expression template encoding an open reading frame operably linked to a promoter element for a DNA-dependent RNA polymerase. In these other aspects, the CFPS reaction mixture can also include a DNA-dependent RNA polymerase to direct transcription of an RNA translation template encoding the open reading frame. In these other aspects, additional NTP's and divalent cation cofactor can be included in the CFPS reaction mixture

A “secondary reaction mixture,” which refers to a solution containing the reagents necessary to carry out an enzyme-mediated biosynthetic steps, typically includes a feedstock that reacts in the presence of the enzyme to produce a final or intermediate product in the metabolic or biosynthetic pathway of interest. A secondary reaction mixture may optionally contain a cofactor, e.g. coenzyme-A, nicotinamide adenine dinucleotide (NAD), adenosine triphosphate (ATP), or a buffer. As used herein, “feedstock” refers to a chemical or material used to perform a reaction. In some embodiments, feedstocks comprise carbon-containing chemicals or compounds. In some embodiments, feedstocks comprise glucose.

The polynucleotide sequences contemplated herein may be present in expression vectors. For example, the vectors may comprise: (a) a polynucleotide encoding an ORF of a protein; (b) a polynucleotide that expresses an RNA that directs RNA-mediated binding, nicking, and/or cleaving of a target DNA sequence; and both (a) and (b). The polynucleotide present in the vector may be operably linked to a prokaryotic or eukaryotic promoter. “Operably linked” refers to the situation in which a first nucleic acid sequence is placed in a functional relationship with a second nucleic acid sequence. For instance, a promoter is operably linked to a coding sequence if the promoter affects the transcription or expression of the coding sequence. Operably linked DNA sequences may be in close proximity or contiguous and, where necessary to join two protein coding regions, in the same reading frame. Vectors contemplated herein may comprise a heterologous promoter (e.g., a eukaryotic or prokaryotic promoter) operably linked to a polynucleotide that encodes a protein. A “heterologous promoter” refers to a promoter that is not the native or endogenous promoter for the protein or RNA that is being expressed. Vectors as disclosed herein may include plasmid vectors.

As used herein, “expression” refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

In certain exemplary embodiments, vectors such as, for example, expression vectors, containing a nucleic acid encoding one or more rRNAs or reporter polypeptides and/or proteins described herein are provided. As used herein, the term “vector” refers to a nucleic acid molecule capable of transporting another nucleic acid to which it has been linked. One type of vector is a “plasmid,” which refers to a circular double stranded DNA loop into which additional DNA segments can be ligated. Such vectors are referred to herein as “expression vectors.” In general, expression vectors of utility in recombinant DNA techniques are often in the form of plasmids. In the present specification, “plasmid” and “vector” can be used interchangeably. However, the disclosed methods and compositions are intended to include such other forms of expression vectors, such as viral vectors (e.g., replication defective retroviruses, adenoviruses and adeno-associated viruses), which serve equivalent functions.

In certain exemplary embodiments, the recombinant expression vectors comprise a nucleic acid sequence (e.g., a nucleic acid sequence encoding one or more rRNAs or reporter polypeptides and/or proteins described herein) in a form suitable for expression of the nucleic acid sequence in one or more of the methods described herein, which means that the recombinant expression vectors include one or more regulatory sequences which is operatively linked to the nucleic acid sequence to be expressed. Within a recombinant expression vector, “operably linked” is intended to mean that the nucleotide sequence encoding one or more rRNAs or reporter polypeptides and/or proteins described herein is linked to the regulatory sequence(s) in a manner which allows for expression of the nucleotide sequence (e.g., in an in vitro transcription and/or translation system). The term “regulatory sequence” is intended to include promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990).

Oligonucleotides and polynucleotides may optionally include one or more non-standard nucleotide(s), nucleotide analog(s) and/or modified nucleotides. Examples of modified nucleotides include, but are not limited to diaminopurine, S2T, 5-fluorouracil, 5-bromouracil, 5-chlorouracil, 5-iodouracil, hypoxanthine, xantine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-D46-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, 2,6-diaminopurine and the like. Nucleic acid molecules may also be modified at the base moiety (e.g., at one or more atoms that typically are available to form a hydrogen bond with a complementary nucleotide and/or at one or more atoms that are not typically capable of forming a hydrogen bond with a complementary nucleotide), sugar moiety or phosphate backbone.

The terms “polynucleotide,” “polynucleotide sequence,” “nucleic acid” and “nucleic acid sequence” refer to a nucleotide, oligonucleotide, polynucleotide (which terms may be used interchangeably), or any fragment thereof. These phrases also refer to DNA or RNA of genomic, natural, or synthetic origin (which may be single-stranded or double-stranded and may represent the sense or the antisense strand).

Regarding polynucleotide sequences, the terms “percent identity” and “% identity” refer to the percentage of residue matches between at least two polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences. Percent identity for a nucleic acid sequence may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST), which is available from several sources, including the NCBI, Bethesda, Md., at its website. The BLAST software suite includes various sequence analysis programs including “blastn,” that is used to align a known polynucleotide sequence with other polynucleotide sequences from a variety of databases. Also available is a tool called “BLAST 2 Sequences” that is used for direct pairwise comparison of two nucleotide sequences. “BLAST 2 Sequences” can be accessed and used interactively at the NCBI website. The “BLAST 2 Sequences” tool can be used for both blastn and blastp (discussed above).

Regarding polynucleotide sequences, percent identity may be measured over the length of an entire defined polynucleotide sequence, for example, as defined by a particular SEQ ID number, or may be measured over a shorter length, for example, over the length of a fragment taken from a larger, defined sequence, for instance, a fragment of at least 20, at least 30, at least 40, at least 50, at least 70, at least 100, or at least 200 contiguous nucleotides. Such lengths are exemplary only, and it is understood that any fragment length supported by the sequences shown herein, in the tables, figures, or Sequence Listing, may be used to describe a length over which percentage identity may be measured.

Regarding polynucleotide sequences, “variant,” “mutant,” or “derivative” may be defined as a nucleic acid sequence having at least 50% sequence identity to the particular nucleic acid sequence over a certain length of one of the nucleic acid sequences using blastn with the “BLAST 2 Sequences” tool available at the National Center for Biotechnology Information's website. (See Tatiana A. Tatusova, Thomas L. Madden (1999), “Blast 2 sequences—a new tool for comparing protein and nucleotide sequences”, FEMS Microbiol Lett. 174:247-250). Such a pair of nucleic acids may show, for example, at least 60%, at least 70%, at least 80%, at least 85%, at least 90%, at least 91%, at least 92%, at least 93%, at least 94%, at least 95%, at least 96%, at least 97%, at least 98%, or at least 99% or greater sequence identity over a certain defined length.

Nucleic acid sequences that do not show a high degree of identity may nevertheless encode similar amino acid sequences due to the degeneracy of the genetic code where multiple codons may encode for a single amino acid. It is understood that changes in a nucleic acid sequence can be made using this degeneracy to produce multiple nucleic acid sequences that all encode substantially the same protein. For example, polynucleotide sequences as contemplated herein may encode a protein and may be codon-optimized for expression in a particular host. In the art, codon usage frequency tables have been prepared for a number of host organisms including humans, mouse, rat, pig, E. coli, plants, and other host cells.

A “recombinant nucleic acid” is a sequence that is not naturally occurring or has a sequence that is made by an artificial combination of two or more otherwise separated segments of sequence. This artificial combination is often accomplished by chemical synthesis or, more commonly, by the artificial manipulation of isolated segments of nucleic acids, e.g., by genetic engineering techniques known in the art. The term recombinant includes nucleic acids that have been altered solely by addition, substitution, or deletion of a portion of the nucleic acid. Frequently, a recombinant nucleic acid may include a nucleic acid sequence operably linked to a promoter sequence. Such a recombinant nucleic acid may be part of a vector that is used, for example, to transform a cell.

The nucleic acids disclosed herein may be “substantially isolated or purified.” The term “substantially isolated or purified” refers to a nucleic acid that is removed from its natural environment, and is at least 60% free, preferably at least 75% free, and more preferably at least 90% free, even more preferably at least 95% free from other components with which it is naturally associated.

### Peptides, Polypeptides, Proteins, and Synthesis Methods

As used herein, the terms “peptide,” “polypeptide,” and “protein,” refer to molecules comprising a chain a polymer of amino acid residues joined by amide linkages. The term “amino acid residue,” includes but is not limited to amino acid residues contained in the group consisting of alanine (Ala or A), cysteine (Cys or C), aspartic acid (Asp or D), glutamic acid (Glu or E), phenylalanine (Phe or F), glycine (Gly or G), histidine (His or H), isoleucine (Ile or I), lysine (Lys or K), leucine (Leu or L), methionine (Met or M), asparagine (Asn or N), proline (Pro or P), glutamine (Gln or Q), arginine (Arg or R), serine (Ser or S), threonine (Thr or T), valine (Val or V), tryptophan (Trp or W), and tyrosine (Tyr or Y) residues. The term “amino acid residue” also may include nonstandard or unnatural amino acids. The term “amino acid residue” may include alpha-, beta-, gamma-, and delta-amino acids.

In some embodiments, the term “amino acid residue” may include nonstandard or unnatural amino acid residues contained in the group consisting of homocysteine, 2-Aminoadipic acid, N-Ethylasparagine, 3-Aminoadipic acid, Hydroxylysine, β-alanine, β-Amino-propionic acid, allo-Hydroxylysine acid, 2-Aminobutyric acid, 3-Hydroxyproline, 4-Aminobutyric acid, 4-Hydroxyproline, piperidinic acid, 6-Aminocaproic acid, Isodesmosine, 2-Aminoheptanoic acid, allo-Isoleucine, 2-Aminoisobutyric acid, N-Methylglycine, sarcosine, 3-Aminoisobutyric acid, N-Methylisoleucine, 2-Aminopimelic acid, 6-N-Methyllysine, 2,4-Diaminobutyric acid, N-Methylvaline, Desmosine, Norvaline, 2,2′-Diaminopimelic acid, Norleucine, 2,3-Diaminopropionic acid, Ornithine, and N-Ethylglycine. The term “amino acid residue” may include L isomers or D isomers of any of the aforementioned amino acids.

Other examples of nonstandard or unnatural amino acids include, but are not limited, to a p-acetyl-L-phenylalanine, a p-iodo-L-phenylalanine, an O-methyl-L-tyrosine, a p-propargyloxyphenylalanine, a p-propargyl-phenylalanine, an L-3-(2-naphthyl)alanine, a 3-methyl-phenylalanine, an O-4-allyl-L-tyrosine, a 4-propyl-L-tyrosine, a tri-O-acetyl-GlcNAcpp-serine, an L-Dopa, a fluorinated phenylalanine, an isopropyl-L-phenylalanine, a p-azido-L-phenylalanine, a p-acyl-L-phenylalanine, a p-benzoyl-L-phenylalanine, an L-phosphoserine, a phosphonoserine, a phosphonotyrosine, a p-bromophenylalanine, a p-amino-L-phenylalanine, an isopropyl-L-phenylalanine, an unnatural analogue of a tyrosine amino acid; an unnatural analogue of a glutamine amino acid; an unnatural analogue of a phenylalanine amino acid; an unnatural analogue of a serine amino acid; an unnatural analogue of a threonine amino acid; an unnatural analogue of a methionine amino acid; an unnatural analogue of a leucine amino acid; an unnatural analogue of a isoleucine amino acid; an alkyl, aryl, acyl, azido, cyano, halo, hydrazine, hydrazide, hydroxyl, alkenyl, alkynl, ether, thiol, sulfonyl, seleno, ester, thioacid, borate, boronate, 20ufa20hor, phosphono, phosphine, heterocyclic, enone, imine, aldehyde, hydroxylamine, keto, or amino substituted amino acid, or a combination thereof; an amino acid with a photoactivatable cross-linker; a spin-labeled amino acid; a fluorescent amino acid; a metal binding amino acid; a metal-containing amino acid; a radioactive amino acid; a photocaged and/or photoisomerizable amino acid; a biotin or biotin-analogue containing amino acid; a keto containing amino acid; an amino acid comprising polyethylene glycol or polyether; a heavy atom substituted amino acid; a chemically cleavable or photocleavable amino acid; an amino acid with an elongated side chain; an amino acid containing a toxic group; a sugar substituted amino acid; a carbon-linked sugar-containing amino acid; a redox-active amino acid; an α-hydroxy containing acid; an amino thio acid; an α,α disubstituted amino acid; a β-amino acid; a γ-amino acid, a cyclic amino acid other than proline or histidine, and an aromatic amino acid other than phenylalanine, tyrosine or tryptophan.

As used herein, a “peptide” is defined as a short polymer of amino acids, of a length typically of 20 or less amino acids, and more typically of a length of 12 or less amino acids (Garrett & Grisham, Biochemistry, 2nd edition, 1999, Brooks/Cole, 110). In some embodiments, a peptide as contemplated herein may include no more than about 2, 3, 4, 5, 6, 7, 8, 9, 10, 11, 12, 13, 14, 15, 16, 17, 18, 19, or 20 amino acids. A polypeptide, also referred to as a protein, is typically of length>100 amino acids (Garrett & Grisham, Biochemistry, 2nd edition, 1999, Brooks/Cole, 110). A polypeptide, as contemplated herein, may comprise, but is not limited to, 100, 101, 102, 103, 104, 105, about 110, about 120, about 130, about 140, about 150, about 160, about 170, about 180, about 190, about 200, about 210, about 220, about 230, about 240, about 250, about 275, about 300, about 325, about 350, about 375, about 400, about 425, about 450, about 475, about 500, about 525, about 550, about 575, about 600, about 625, about 650, about 675, about 700, about 725, about 750, about 775, about 800, about 825, about 850, about 875, about 900, about 925, about 950, about 975, about 1000, about 1100, about 1200, about 1300, about 1400, about 1500, about 1750, about 2000, about 2250, about 2500 or more amino acid residues.

A peptide as contemplated herein may be further modified to include non-amino acid moieties. Modifications may include but are not limited to acylation (e.g., O-acylation (esters), N-acylation (amides), S-acylation (thioesters)), acetylation (e.g., the addition of an acetyl group, either at the N-terminus of the protein or at lysine residues), formylation lipoylation (e.g., attachment of a lipoate, a C8 functional group), myristoylation (e.g., attachment of myristate, a C14 saturated acid), palmitoylation (e.g., attachment of palmitate, a C16 saturated acid), alkylation (e.g., the addition of an alkyl group, such as an methyl at a lysine or arginine residue), isoprenylation or prenylation (e.g., the addition of an isoprenoid group such as farnesol or geranylgeraniol), amidation at C-terminus, glycosylation (e.g., the addition of a glycosyl group to either asparagine, hydroxylysine, serine, or threonine, resulting in a glycoprotein). Distinct from glycation, which is regarded as a nonenzymatic attachment of sugars, polysialylation (e.g., the addition of polysialic acid), glypiation (e.g., glycosylphosphatidylinositol (GPI) anchor formation, hydroxylation, iodination (e.g., of thyroid hormones), and phosphorylation (e.g., the addition of a phosphate group, usually to serine, tyrosine, threonine or histidine).

A modified amino acid sequence that is disclosed herein may include a deletion in one or more amino acids. As utilized herein, a “deletion” means the removal of one or more amino acids relative to the native amino acid sequence. The modified amino acid sequences that are disclosed herein may include an insertion of one or more amino acids. As utilized herein, an “insertion” means the addition of one or more amino acids to a native amino acid sequence. The modified amino acid sequences that are disclosed herein may include a substitution of one or more amino acids. As utilized herein, a “substitution” means replacement of an amino acid of a native amino acid sequence with an amino acid that is not native to the amino acid sequence.

A “variant,” “mutant,” or “derivative” of a reference polypeptide sequence may include a deletion relative to the reference polypeptide sequence.

Regarding proteins, “fragment” is a portion of an amino acid sequence which is identical in sequence to but shorter in length than a reference sequence. A fragment may comprise up to the entire length of the reference sequence, minus at least one amino acid residue. For example, a fragment may comprise from 5 to 1000 contiguous amino acid residues of a reference polypeptide, respectively. In some embodiments, a fragment may comprise at least 5, 10, 15, 20, 25, 30, 40, 50, 60, 70, 80, 90, 100, 150, 250, or 500 contiguous amino acid residues of a reference polypeptide. Fragments may be preferentially selected from certain regions of a molecule. The term “at least a fragment” encompasses the full-length polypeptide. A fragment may include an N-terminal truncation, a C-terminal truncation, or both truncations relative to the full-length protein. A “variant,” “mutant,” or “derivative” of a reference polypeptide sequence may include a fragment of the reference polypeptide sequence.

Regarding proteins, the words “insertion” and “addition” refer to changes in an amino acid sequence resulting in the addition of one or more amino acid residues. An insertion or addition may refer to 1, 2, 3, 4, 5, 10, 20, 30, 40, 50, 60, 70, 80, 90, 100, 150, 200, or more amino acid residues. A “variant,” “mutant,” or “derivative” of a reference polypeptide sequence may include an insertion or addition relative to the reference polypeptide sequence. A variant of a protein may have N-terminal insertions, C-terminal insertions, internal insertions, or any combination of N-terminal insertions, C-terminal insertions, and internal insertions.

Regarding proteins, the phrases “percent identity” and “% identity,” refer to the percentage of residue matches between at least two amino acid sequences aligned using a standardized algorithm. Methods of amino acid sequence alignment are well-known. Some alignment methods take into account conservative amino acid substitutions. Such conservative substitutions, explained in more detail below, generally preserve the charge and hydrophobicity at the site of substitution, thus preserving the structure (and therefore function) of the polypeptide. Percent identity for amino acid sequences may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST), which is available from several sources, including the NCBI, Bethesda, Md., at its website. The BLAST software suite includes various sequence analysis programs including “blastp,” that is used to align a known amino acid sequence with other amino acids sequences from a variety of databases.

Regarding proteins, percent identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

Regarding proteins, the amino acid sequences of variants, mutants, or derivatives as contemplated herein may include conservative amino acid substitutions relative to a reference amino acid sequence. For example, a variant, mutant, or derivative protein may include conservative amino acid substitutions relative to a reference molecule. “Conservative amino acid substitutions” are those substitutions that are a substitution of an amino acid for a different amino acid where the substitution is predicted to interfere least with the properties of the reference polypeptide. In other words, conservative amino acid substitutions substantially conserve the structure and the function of the reference polypeptide. The following table provides a list of exemplary conservative amino acid substitutions which are contemplated herein:

| Original | Conservative |
|---|---|
| Residue | Substitution |
| Ala | Gly, Ser |
| Arg | His, Lys |
| Asn | Asp, Gln, His |
| Asp | Asn, Glu |
| Cys | Ala, Ser |
| Gln | Asn, Glu, His |
| Glu | Asp, Gln, His |
| Gly | Ala |
| His | Asn, Arg, Gln, Glu |
| Ile | Leu, Val |
| Leu | Ile, Val |
| Lys | Arg, Gln, Glu |
| Met | Leu, Ile |
| Phe | His, Met, Leu, Trp, Tyr |
| Ser | Cys, Thr |
| Thr | Ser, Val |
| Trp | Phe, Tyr |
| Tyr | His, Phe, Trp |
| Val | Ile, Leu, Thr |

Conservative amino acid substitutions generally maintain (a) the structure of the polypeptide backbone in the area of the substitution, for example, as a beta sheet or alpha helical conformation, (b) the charge or hydrophobicity of the molecule at the site of the substitution, and/or (c) the bulk of the side chain. Non-conservative amino acids typically disrupt (a) the structure of the polypeptide backbone in the area of the substitution, for example, as a beta sheet or alpha helical conformation, (b) the charge or hydrophobicity of the molecule at the site of the substitution, and/or (c) the bulk of the side chain.

The disclosed proteins, mutants, or variants, described herein may have one or more functional or biological activities exhibited by a reference polypeptide (e.g., one or more functional or biological activities exhibited by wild-type protein). In some embodiments, the activity of the variant or mutant protein (e.g., a modified NGT as disclosed herein) may have an activity that is enhanced, as compared to a comparable wild-type or control NGT enzyme, or may have an alternative or a modified activity as compared to a comparable or wild-type or control NGT enzyme.

### Cell-Free Protein Synthesis (CFPS)

The components, systems, and methods disclosed herein may be applied to, or adapted to cell-free protein synthesis methods as known in the art. See, for example, U.S. Pat. Nos. 5,478,730; 5,556,769; 5,665,563; 6,168,931; 6,548,276; 6,869,774; 6,994,986; 7,118,883; 7,186,525; 7,189,528; 7,235,382; 7,338,789; 7,387,884; 7,399,610; 7,776,535; 7,817,794; 8,703,471; 8,298,759; 8,715,958; 8,734,856; 8,999,668; and 9,005,920. See also U.S. Published Application Nos. 2018/0016614, 2018/0016612, 2016/0060301, 2015-0259757, 2014/0349353, 2014-0295492, 2014-0255987, 2014-0045267, 2012-0171720, 2008-0138857, 2007-0154983, 2005-0054044, and 2004-0209321. See also U.S Published Application Nos. 2005-0170452; 2006-0211085; 2006-0234345; 2006-0252672; 2006-0257399; 2006-0286637; 2007-0026485; 2007-0178551. See also Published PCT International Application Nos. 2003/056914; 2004/013151; 2004/035605; 2006/102652; 2006/119987; and 2007/120932. See also Jewett, M. C., Hong, S. H., Kwon, Y. C., Martin, R. W., and Des Soye, B. J. 2014, “Methods for improved in vitro protein synthesis with proteins containing non-standard amino acids,” U.S. Patent Application Ser. No. 62/044,221; Jewett, M. C., Hodgman, C. E., and Gan, R. 2013, “Methods for yeast cell-free protein synthesis,” U.S. Patent Application Ser. No. 61/792,290; Jewett, M. C., J. A. Schoborg, and C. E. Hodgman. 2014, “Substrate Replenishment and Byproduct Removal Improve Yeast Cell-Free Protein Synthesis,” U.S. Patent Application Ser. No. 61/953,275; and Jewett, M. C., Anderson, M. J., Stark, J. C., Hodgman, C. E. 2015, “Methods for activating natural energy metabolism for improved yeast cell-free protein synthesis,” U.S. Patent Application Ser. No. 62/098,578. See also Guarino, C., & DeLisa, M. P. (2012). A prokaryote-based cell-free translation system that efficiently synthesizes glycoproteins. Glycobiology, 22(5), 596-601. The contents of all of these references are incorporated in the present application by reference in their entireties.

As described above, in some embodiments, a “CFPS reaction mixture” typically may contain a crude or partially-purified cell extract (e.g., a yeast or bacterial extract), an RNA translation template, and a suitable reaction buffer for promoting cell-free protein synthesis from the RNA translation template. In some aspects, the CFPS reaction mixture can include exogenous RNA translation template. In other aspects, the CFPS reaction mixture can include a DNA expression template encoding an open reading frame operably linked to a promoter element for a DNA-dependent RNA polymerase. In these other aspects, the CFPS reaction mixture can also include a DNA-dependent RNA polymerase to direct transcription of an RNA translation template encoding the open reading frame. In these other aspects, additional NTP's and divalent cation cofactor can be included in the CFPS reaction mixture. A reaction mixture is referred to as complete if it contains all reagents necessary to enable the reaction, and incomplete if it contains only a subset of the necessary reagents. It will be understood by one of ordinary skill in the art that reaction components are routinely stored as separate solutions, each containing a subset of the total components, for reasons of convenience, storage stability, or to allow for application-dependent adjustment of the component concentrations, and that reaction components are combined prior to the reaction to create a complete reaction mixture. Furthermore, it will be understood by one of ordinary skill in the art that reaction components are packaged separately for commercialization and that useful commercial kits may contain any subset of the reaction components of the invention.

The disclosed cell-free protein synthesis systems may utilize components that are crude and/or that are at least partially isolated and/or purified. As used herein, the term “crude” may mean components obtained by disrupting and lysing cells and, at best, minimally purifying the crude components from the disrupted and lysed cells, for example by centrifuging the disrupted and lysed cells and collecting the crude components from the supernatant and/or pellet after centrifugation. The term “isolated or purified” refers to components that are removed from their natural environment, and are at least 60% free, preferably at least 75% free, and more preferably at least 90% free, even more preferably at least 95% free from other components with which they are naturally associated.

In some embodiments, CFPS reactions include a crude or partially-purified cell extract. In some embodiments, the cells used to derive the crude or partially purified extract may be selected based on the presence or absence of specific endogenous biochemical pathways, and/or engineered biochemical pathways. For example, cells that direct carbon flux, prevent or minimize side product formation, and prevent or minimize promiscuous background activity may be advantageous as compared to other cells. In some embodiments, the cell is a prokaryotic cell (e.g., bacterial cell) or a eukaryotic cell (e.g., a yeast cell). In some embodiments, the cell is a prokaryotic cell and comprises and E. coli cell. In some embodiments, the E. coli cell comprises a modified E. coli cell, such as BL21, JST07, MB263, MP263sucD, and JC01. In some embodiments, the E. coli cell comprises JST07.

As used herein, “translation template” for a polypeptide refers to an RNA product of transcription from an expression template that can be used by ribosomes to synthesize polypeptides or proteins.

The term “reaction mixture,” as used herein, refers to a solution containing reagents necessary to carry out a given reaction. A reaction mixture is referred to as complete if it contains all reagents necessary to perform the reaction. Components for a reaction mixture may be stored separately in separate container, each containing one or more of the total components. Components may be packaged separately for commercialization and useful commercial kits may contain one or more of the reaction components for a reaction mixture.

For example, a CFPS reaction mixture may include an expression template, a translation template, or both an expression template and a translation template. The expression template serves as a substrate for transcribing at least one RNA that can be translated into a sequence defined biopolymer (e.g., a polypeptide or protein). The translation template is an RNA product that can be used by ribosomes to synthesize the sequence defined biopolymer. In certain embodiments the platform comprises both the expression template and the translation template. In certain specific embodiments, the reaction mixture may comprise a coupled transcription/translation (“Tx/Tl”) system where synthesis of translation template and a sequence defined biopolymer from the same cellular extract.

The CFPS reaction mixture may comprise one or more polymerases capable of generating a translation template from an expression template. The polymerase may be supplied exogenously or may be supplied from the organism used to prepare the extract. In certain specific embodiments, the polymerase is expressed from a plasmid present in the organism used to prepare the extract and/or an integration site in the genome of the organism used to prepare the extract.

Altering the physicochemical environment of the CFPS reaction to better mimic the cytoplasm can improve protein synthesis activity. The following parameters can be considered alone or in combination with one or more other components to improve robust CFPS reaction platforms based upon crude cellular extracts.

The temperature may be any temperature suitable for CFPS. Temperature may be in the general range from about 10° C. to about 40° C., including intermediate specific ranges within this general range, include from about 15° C. to about 35° C., from about 15° C. to about 30° C., from about 15° C. to about 25° C. In certain aspects, the reaction temperature can be about 15° C., about 16° C., about 17° C., about 18° C., about 19° C., about 20° C., about 21° C., about 22° C., about 23° C., about 24° C., about 25° C.

The reaction mixture may include any organic anion suitable for CFPS. In certain aspects, the organic anions can be glutamate, acetate, among others. In certain aspects, the concentration for the organic anions is independently in the general range from about 0 mM to about 200 mM, including intermediate specific values within this general range, such as about 0 mM, about 10 mM, about 20 mM, about 30 mM, about 40 mM, about 50 mM, about 60 mM, about 70 mM, about 80 mM, about 90 mM, about 100 mM, about 110 mM, about 120 mM, about 130 mM, about 140 mM, about 150 mM, about 160 mM, about 170 mM, about 180 mM, about 190 mM and about 200 mM, among others.

The reaction mixture may include any halide anion suitable for CFPS. In certain aspects the halide anion can be chloride, bromide, iodide, among others. A preferred halide anion is chloride. Generally, the concentration of halide anions, if present in the reaction, is within the general range from about 0 mM to about 200 mM, including intermediate specific values within this general range, such as those disclosed for organic anions generally herein.

The reaction mixture may include any organic cation suitable for CFPS. In certain aspects, the organic cation can be a polyamine, such as spermidine or putrescine, among others. Preferably polyamines are present in the CFPS reaction. In certain aspects, the concentration of organic cations in the reaction can be in the general about 0 mM to about 3 mM, about 0.5 mM to about 2.5 mM, about 1 mM to about 2 mM. In certain aspects, more than one organic cation can be present.

The reaction mixture may include any inorganic cation suitable for CFPS. For example, suitable inorganic cations can include monovalent cations, such as sodium, potassium, lithium, among others; and divalent cations, such as magnesium, calcium, manganese, among others. In certain aspects, the inorganic cation is magnesium. In such aspects, the magnesium concentration can be within the general range from about 1 mM to about 50 mM, including intermediate specific values within this general range, such as about 1 mM, about 2 mM, about 3 mM, about 5 mM, about 6 mM, about 7 mM, about 8 mM, about 9 mM, about 10 mM, among others. In preferred aspects, the concentration of inorganic cations can be within the specific range from about 4 mM to about 9 mM and more preferably, within the range from about 5 mM to about 7 mM.

The reaction mixture may include endogenous NTPs (i.e., NTPs that are present in the cell extract) and or exogenous NTPs (i.e., NTPs that are added to the reaction mixture). In certain aspects, the reaction use ATP, GTP, CTP, and UTP. In certain aspects, the concentration of individual NTPs is within the range from about 0.1 mM to about 2 mM.

The reaction mixture may include any alcohol suitable for CFPS. In certain aspects, the alcohol may be a polyol, and more specifically glycerol. In certain aspects the alcohol is between the general range from about 0% (v/v) to about 25% (v/v), including specific intermediate values of about 5% (v/v), about 10% (v/v) and about 15% (v/v), and about 20% (v/v), among others.

### Secondary Reaction

A “secondary reaction mixture,” as used herein, includes bio-manufacturing reactions comprising a CFPS reaction, or products isolated therefrom, and one or more feedstocks. In some embodiments, additional components are included.

Thus, the secondary reaction involves combining the cell-free protein synthesis reaction mixture and the feedstock, wherein the feedstock reacts in the presence of the one or more enzymes to produce the desired product, or an intermediate of the desired product in the desired product pathway.

By way of example, in some embodiments, the secondary reaction comprise one or more cell-free protein synthesis reaction mixtures comprising one or more enzymes comprising a hexanoic acid pathway, and a feedstock comprising glucose or a product of glycolysis. The feedstock reacts in the presence of the one or more enzymes to produce hexanoic acid or an intermediate of hexanoic acid in the hexanoic acid synthetic pathway. In some embodiments, the one or more enzymes are selected from the group consisting of acetyl-CoA acetyltransferase (ThlA), β-hydroxybutyryl-CoA dehydrogenase (Hbd1), 3-hydroxybutyryl-CoA dehydratase (Crt), trans-enoyl-CoA reductase (Ter), thioesterase 1 (TesA), and combinations thereof. In some embodiments, the intermediate of hexanoic acid in the hexanoic acid synthetic pathway is selected from acetyl-CoA, acetoacetyl-CoA, 3-hydroxybutyryl-CoA, hex-(2E)-enoyl-CoA, and hexanoyl-CoA. Optionally, in some embodiments, one or more of CoA, and NAD+ are added to the secondary reaction.

In some embodiments, the secondary reaction comprise one or more cell-free protein synthesis reaction mixtures comprising one or more enzymes comprising an olivetolic acid pathway, and a feedstock comprising glucose or a product of glycolysis and hexanoyl-CoA. The feedstock reacts in the presence of the one or more enzymes to produce olivetolic acid or an intermediate of olivetolic acid in the olivetolic acid synthetic pathway. In some embodiments, the one or more enzymes are selected from the group consisting of acetyl coenzyme A carboxylase (AccBCDA) and biotin-[acetyl-CoA-carboxylase]ligase (BirA), 3,5,7-Trioxododecanoyl-CoA synthase (TKS), olivtolic acid cyclase (OAC), and combinations and combinations thereof. In some embodiments, the intermediate of olivetolic acid in the olivetolic acid synthetic pathway is selected from acetyl-CoA, malonyl-CoA, hexanoyl-CoA, and 3,5,7-trioxododecanoyl-CoA. Optionally, in some embodiments, cerulenin is added to the secondary reaction.

In some embodiments, the secondary reaction comprise one or more cell-free protein synthesis reaction mixtures comprising enzymes comprising a cannabigerolic acid pathway, and a feedstock comprising glucose or a product of glycolysis and olivetolic acid. The feedstock reacts in the presence of the one or more enzymes to produce cannabigerolic acid or an intermediate of cannabigerolic acid in the cannabigerolic acid synthetic pathway. In some embodiments, the one or more enzymes are selected from the group consisting of acetyl-CoA acetyltransferase (AtoB), 3-hydroxy-3-methyl-glutaryl-CoA synthase (HMGS), 3-hydroxy-3-methyl-glutaryl-coenzyme A reductase (HMGR), mevalonate kinase (Mk), phosphomevalonate kinase (PMK), pyrophosphomevalonate decarboxylase (PMD), isopentenyl pyrophosphate isomerase (IDI), geranyl diphosphate synthase (GPPS), and prenyltransferase NphB7, and combinations thereof. In some embodiments, the intermediate of cannabigerolic acid in the cannabigerolic acid synthetic pathway is selected from acetyl-CoA, mevalonate, and geranyl pyrophosphate.

In certain exemplary embodiments, one or more of the methods described herein are performed in a vessel, e.g., a single, vessel. The term “vessel,” as used herein, refers to any container suitable for holding on or more of the reactants (e.g., for use in one or more transcription, translation, and/or secondary reaction steps) described herein. Examples of vessels include, but are not limited to, a microtitre plate, a test tube, a microfuge tube, a beaker, a flask, a multi-well plate, a cuvette, a flow system, a microfiber, a microscope slide and the like.

### Applications and Advantages

The systems, methods, and components disclosed herein find use in numerous applications and advantages. Non-limiting examples include the following:

High-throughput in vitro prototyping of reverse ß-oxidation, isoprenoid and cannabinoid biosynthesis pathways; in vitro biomanufacturing of olivetolic acid and CBGA from glucose alone; the use of discovered optimal enzyme combinations for in vitro cannabinoid precursor synthesis and screening of rare CBGA cyclases with the produced products; the use of specific reverse ß-oxidation enzyme combinations for the synthesis of fatty acids and fatty acid derivatives; the synthesis of rare and/or unnatural cannabinoids in cell-free extracts; debugging of in vitro glycolytic flux of CFME reactions; and cell-free biomanufacturing of cannabinoids.

Both chemical and in vivo biosynthesis strategies have led to low yields of the final cannabinoid products. The extract-based in vitro biomanufacturing platform can overcome some of the bottlenecks of both these approaches and already produces olivetolic acid at high yield.

The open reaction environment allows for the addition of non-natural building blocks that could lead to the formation of non-natural cannabinoids.

The high-throughput screening platform established for both hexanoyl-CoA and olivetolic acid biosynthesis allows for the fast screening of enzyme variants that allow for the accommodation of non-natural building blocks as well as for the further optimization of the complete pathway.

The use of cerulenin as a specific inhibitor of fatty acid biosynthesis, but notably not reverse β-oxidation allows for high pools of malonyl-CoA, one of the limiting substrates in the biosynthesis pathway of olivetolic acid. Our strategy prevents the depletion of that crucial intermediate.

In situ synthesis of high amount of CBGA will allow for the screening of uncharacterized terminal cyclases, which can potentially be produced in high-throughput using cell-free protein synthesis, to synthesize some of the low abundant cannabinoids. Identified cyclases can then easily be integrated into the biomanufacturing platform and would not have to undergo long in vivo strain development cycles.

Our cell-free platform allows for the prototyping and optimization of three relevant metabolic pathways in high-throughput using plate-based analysis tools. It is the first in vitro platform that synthesizes hexanoyl-CoA and OA at high yields and has therefore the potential to overcome the common bottlenecks in cannabinoid biosynthesis. The platform may additionally help identify enzyme candidates that can be used to overcome bottlenecks in in vivo systems by rapidly prototyping enzyme homologues or engineered enzyme variants.

There are no commercially available products for cell-free extract based metabolic engineering platforms available. Commercially available cell-free protein synthesis systems do not contain the necessary knockouts needed to direct carbon flux towards the pathways presented here and would have to be adapted drastically for this purpose.

### EXEMPLARY EMBODIMENTS

We developed a cell-free system for making cannibinoids. This is based on using crude extracts and leverages reverse beta-oxidation, which is unique and surprising.

Embodiment 1. A high-throughput prototyping and manufacturing platform for in vitro biosynthesis of fatty acids and fatty acid derivatives using the reverse ß-oxidation comprising one or more of:
(i) combinatorial assembly of pathways in cell-free systems by mixing-and-matching enzymes produced in cell-free protein synthesis;
(ii) the use of genetically modified E. coli strains for extracts optimized in CoA precursor pools and carbon flux in both cell-free protein synthesis and cell-free conversion of glucose to the target product;
(iii) rapid pathway assembly using acoustic liquid handling robotics; and
(iv) rapid plate-based extraction and derivatization methods for GC-MS analysis of the products.

Embodiment 2. The use of cerulenin as a specific inhibitor of fatty acid biosynthesis, but not reverse ß-oxidation used, for example, with the method of embodiment 1. Without wishing to be bound by theory, the short-chain specificity of the used thiolase prevents the C12-fatty acid mimic to enter its active site, while irreversibly inhibiting the 8-keto-acyl-ACP synthase of natural fatty acid biosynthesis, thus preventing depletion of malonyl-CoA pools.

Embodiment 3. The method of embodiment 1 or 2, where cannabinoid precursors are built using reverse-beta oxidation.

Embodiment 4. The method of any of the previous embodiments, used for identifying best sets of enzymes for olivetolic acid and CBGA biosynthesis, or biosynthesis of their derivatives.

Embodiment 5. The method of any of the previous embodiments, where 100s to 1000s of reactions can be assessed in days rather than 10s.

Embodiment 6. The method any of the previous embodiments, used for manufacturing olivetolic acid and CBGA, or their derivatives.

Embodiment 7. The methods any of the previous embodiments, where enzymes are enriched in lysates by cell-free protein synthesis.

Embodiment 8. The methods any of the previous embodiments, where enzymes are enriched in lysates by overexpression in the host cell.

Embodiment 9. The methods any of the previous embodiments, where enzymes are in lysates by overexpression in multiple distinct host cells, including those from different organisms like E. coli and yeast, and then mixed to construct the full pathway.

### EXAMPLES

The following Examples are illustrative and are not intended to limit the scope of the claimed subject matter.

### Example 1

Common methods for cannabinoid synthesis involve extraction out of plants and production in microbial host strains such as Saccharomyces cerevisiae and Escherichia coli. Extraction from plants has proven difficult due to the high similarities between the different cannabinoids and isolation of rare cannabinoids at high enough titers for characterization is extremely challenging.

Implementation of the biosynthetic pathways into Saccharomyces cerevisiae has recently led to successful production of cannabinoids in yeast for the first time. It was shown that both hexanoyl-CoA and olivetolic acid (OA) are the limiting factor for the production of cannabinoids. The highest yield of OA was achieved in a yeast strain supplemented with hexanoic acid at a yield of 1.6 mg/L (compared to the unsupplemented production of 50 mg/L in an in vitro platform). OA was then successfully converted to CBGA (1.4 mg/L) and downstream cannabinoids cannabidiolic acid (CBDA) (4.3 μg/L) and tetrahydrocannabinolic acid (THCA) (2.3 mg/L). This highlights the potential of our in vitro system to overcome this bottleneck by the efficient synthesis of hexanoyl-CoA via the reverse β-oxidation module.

Implementation of a similar β-oxidation module into E. coli (different enzyme combination) has led to the successful production of 10 mg/L OA (80 mg/L when supplemented with hexanoic acid). The conversion of OA to CBGA and further to THCA, CBDA and other cannabinoids has to our knowledge, not been shown in bacteria. These conversions are very challenging in vivo, due to the nature of the prenyltransferase (membrane bound enzyme), the low abundance of geranyl pyrophosphate (GPP) and OA intracellularly and the cannabinoid cyclases (disulfide bonds, glycosylated). In vitro platforms have multiple tools available to overcome these bottlenecks and may therefore be a great tool to not only serve as a great stand-alone biomanufacturing platform, but also to screen large libraries of enzymes to identify candidates that can help in vivo engineering efforts in bacteria.

Valliere et al. recently engineered a soluble prenyltransferase (NphB7) and successfully established an in vitro system with purified enzymes, that produces CBGA, when supplemented with OA (see Valliere, M. A., et al., A cell-free platform for the prenylation of natural products and application to cannabinoid production. Nature Communications, 2019. 10). They show that the isoprenoid branch of cannabinoid biosynthesis can be run efficiently in vitro and are able to produce CBGA at titers of 1.25 g/L in an optimized reaction vessel. The combination of an optimized isoprenoid branch with the optimized hexanoyl-CoA and OA branch we present here has the potential to produce very high yields of CBGA and downstream cannabinoids from glucose as the only carbon source. Our extract-based system lowers the cost drastically compared to the purified system and additionally offers the advantage of very efficient cofactor regeneration and of improved enzyme stability. These two studies in combination show that in vitro biomanufacturing of cannabinoids is a very valid strategy to pursue and can be competitive with in vivo approaches in this field.

### Example 2—A Cell-Free Biomanufacturing Platform for Production of Fatty Acids and Cannabinoids

### 1. High-Throughput in Vitro Platform for Reverse β-Oxidation to Optimize Hexanoic Acid Yields

### 1.1 Selection of an Optimized Extract Background Strain

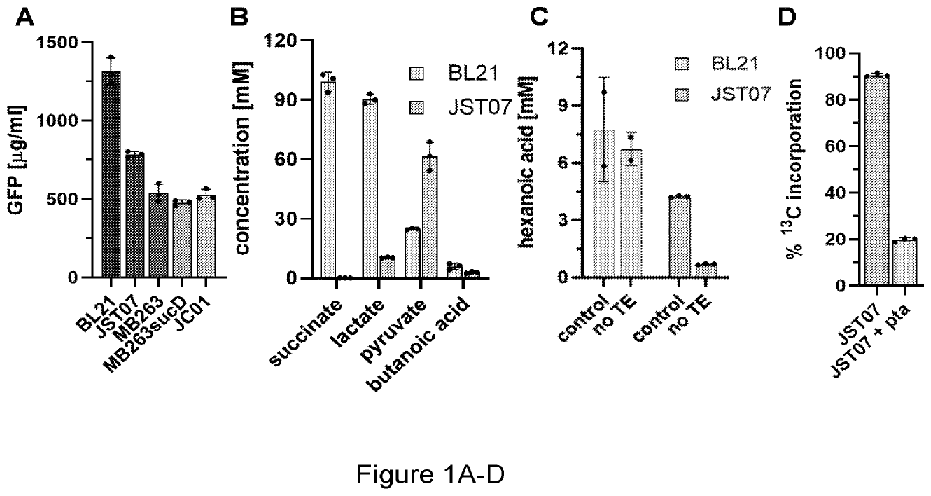

There are significant benefits in picking the appropriate E. coli strain for the targeted metabolic pathway. Knockouts can help direct carbon flux, prevent side product formation and promiscuous background activity of native enzyme homologues of the investigated cascade. We therefore picked a previously published reversal of β-oxidation (r-BOX) enzyme set as our base case to study the effect of knockouts targeting fermentation pathways and thioesterase activity, which both have been shown to improve r-BOX yields in vivo (see Dellomonaco, C., et al., Engineered reversal of the beta-oxidation cycle for the synthesis of fuels and chemicals. Nature, 2011. 476(7360): p. 355-U131; Cheong, S., J. M. Clomburg, and R. Gonzalez, Energy- and carbon-efficient synthesis of functionalized small molecules in bacteria using non-decarboxylative Claisen condensation reactions. Nature Biotechnology, 2016. 34(5): p. 556-561; and Karim, A. S., et al., In vitro prototyping and rapid optimization of biosynthetic enzymes for cell design. Nature Chemical Biology, 2020). MB263 contains knockouts for all the mixed-acid fermentation pathways of E. coli (ΔldhAΔpoxBΔptaΔadhE), JC01 additionally contains a ΔfrdA knockout and JST07 additionally has five known thioesterases (ΔyciA ΔybgC Δydil ΔtesA ΔfadM) and fadE removed. We first optimized growth, harvest and extract preparation of BL21Star(DE3) (from now on BL21*), MB263, JC01 and JST07 for cell-free protein synthesis of sfGFP, as production of high amount of soluble pathway enzymes is crucial for the combinatorial two-pot system (FIG. 1A). Optimized extracts yielded 1312±70 ng/mL for BL21*537±46 ng/mL for MB263, 535±29 ng/mL for JC01 and 785±18 ng/mL for JST07. ¹⁴C-labeled leucine incorporation was used to determine the CFPS yields of the initial set of r-BOX enzymes (Table 2). The rBOX enzymes were then combined in a second step into a functional pathway, metabolism started by the addition of catalytic amounts of cofactors and 120 mM glucose and reaction products were determined by HPLC (for side products and butanoic acid) and GC-MS (for hexanoic acid). BL21* produces a large amount of lactic and succinic acid, while the major side product of JC01 and JST07 was pyruvate (FIG. 1B). Pyruvate is not a dead-end product and can potentially be further converted to acetyl-CoA, which feeds into r-BOX. We next systematically left out one of the pathway enzymes to test for background activity stemming from the native E. coli extract. Both BL21* and JC01 showed significant thioesterase activity, which makes screening for a specific thioesterase activity in this extract background impossible and highlights the need to use tailored extracts in future iPROBE bases in in vitro prototyping approaches (FIG. 1C). Note that the fast testing cycle of this iPROBE approach can additionally serve as a method to prototype different backgrounds before a pathway is fully transplanted in vivo. Quantification of the side products in BL21 indicated that glucose is not the only carbon source used in the CFME reaction and it has previously been described that acetate, which is present in the assay at (give actual number) high concentrations can enter metabolism via the acetate kinase AckA and phosphate acetyltransferase Pta. We tested carbon flux by using ¹³C-labeled glucose and quantified the amount of heavy carbon in hexanoic acid. The knockout of pta in JC01 and JST07 prevented equilibration of the labeled acetyl-CoA originating from glucose into the existing unlabeled acetate pool therefore allowing for carbon balance calculations in those extracts (FIG. 1D). Addition of pta produced in CFPS lead to a similar loss in labeled hexanoic acid as observed in the BL21* strain. We therefore picked JST07 as our platform strain due to the fact that i) only pyruvate accumulated at a significant level as a side product (which may be consumed by more efficient r-BOX variants), ii) the thioesterase knockouts significantly reduced background activity allowing for the screening of specific termination enzymes and iii) the inability of the extract to utilize acetate salts present in the reactions allow for carbon balance calculations.

1.2 A 96-Well Plate Screening Platform for r-BOX Variants Using Acoustic Liquid Handling

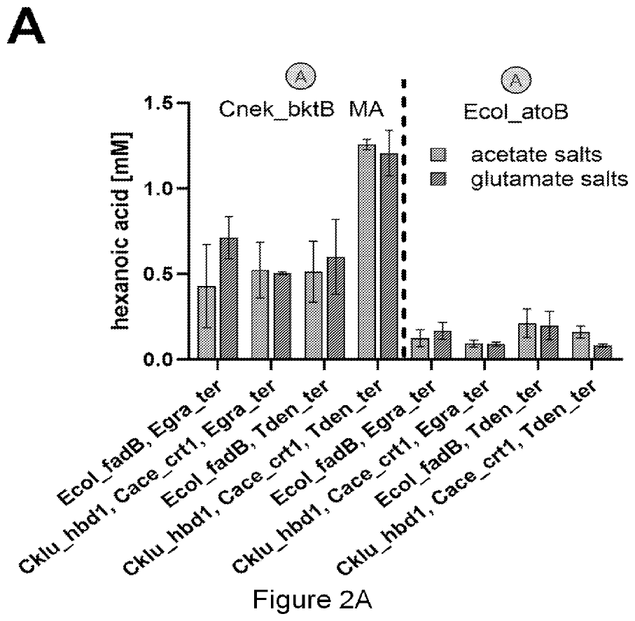

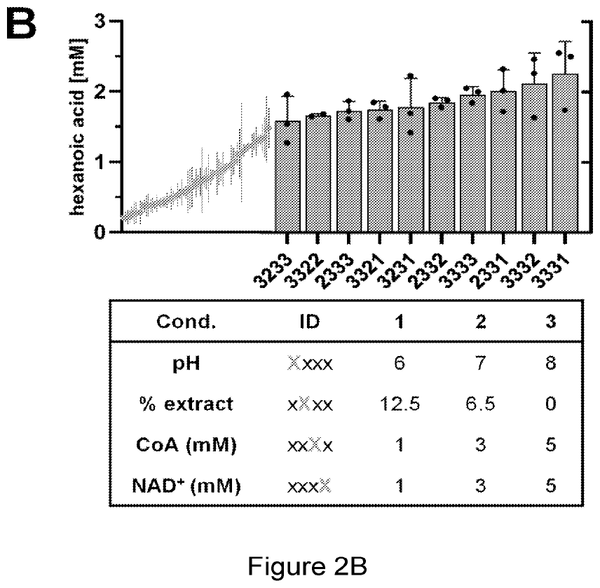

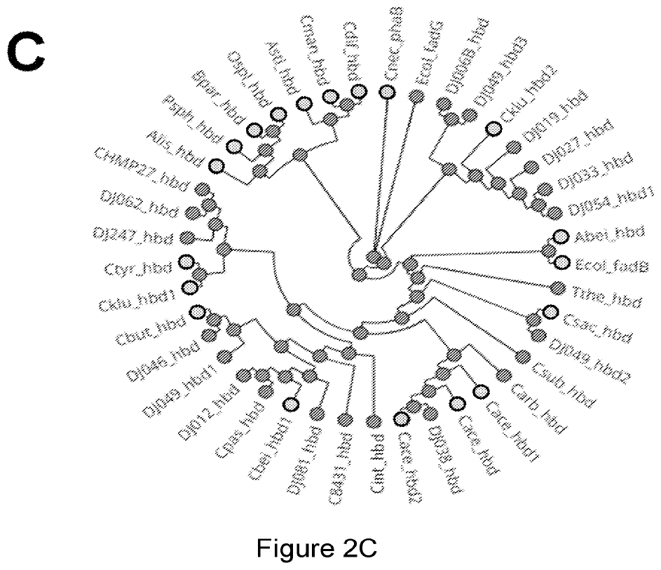

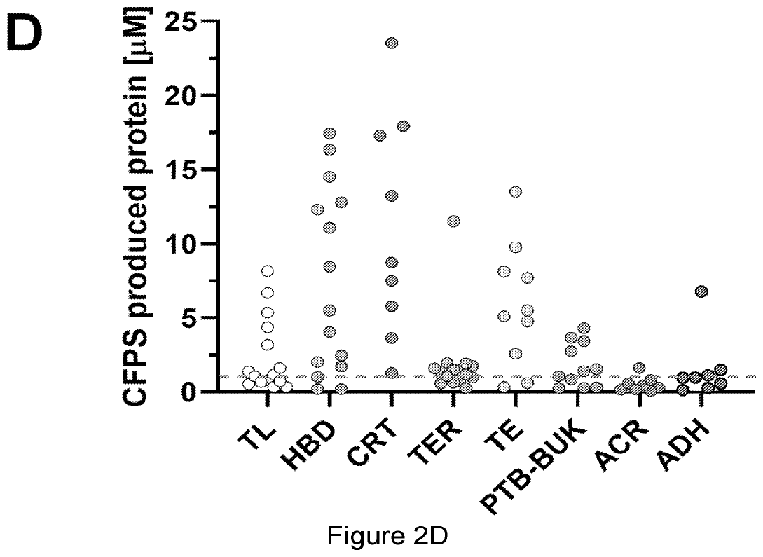

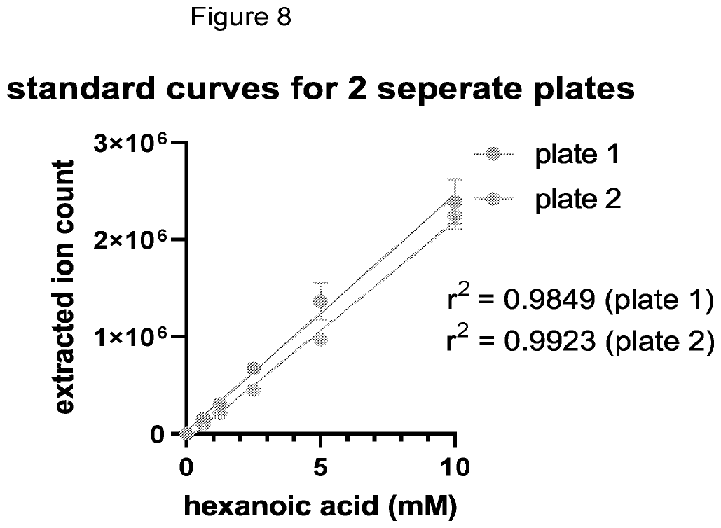

Next, we screened the previously reported functional r-BOX enzymes from previous in vivo (see Cheong, S., J. M. Clomburg, and R. Gonzalez, supra) and in vitro (Karim, A. S., et al., supra) studies in an initial combinatorial CFME set to determine a good starting point to downscale our reactions into a 96-well plate format (FIG. 2A). We additionally tested both acetate and glutamate salts as that had an effect on yields in previous studies resulting in a total of 16 different pathway variants. We could not detect any significant difference between the two salts, most likely due to the inability of the JST07 extract to utilize acetate via Pta. The best combination consisting of Cnek_bktB M158A, Cklu_hbd1, Cace_crt1, Tden_ter and Ecol_tesA produced 1.26±0.3 mM hexanoic acid. Next, we scaled our CFME reactions from 15 mL down to 4 mL reactions and assembled them with an acoustic liquid handling robot into glass coated 96-well plates. Samples were incubated for 20 h and then directly quenched, extracted and derivatized in a 96-well plate format and analyzed via GC-MS. Quantification was done via an on plate standard curve extracted from 4 μL empty reactions (example standard curve FIG. 8). Optimization of CoA, NAD+, pH and amount of fresh extract (i.e. amount of glycolytic enzymes in relation to r-BOX enzymes) added to the CFME reaction resulted in an increase of hexanoic acid yield to 2.3±0.5 mM (FIG. 2B). 5 mM CoA, 3 mM NAD*, 0% fresh extract and pH 7 (condition 2332) were picked as starting point for the combinatorial screen of r-BOX enzymes. We chose pH 7 as it is closer to the intracellular pH of Clostridium autoethanogenum and 3 mM NADY over 1 mM NAD⁺ due to the possibility to switch out our initial termination enzyme with NADH consuming acyl-CoA reductases (ACR) and alcohol dehydrogenases (ADH), which would most likely profit from an increased nicotinamide cofactor pool.

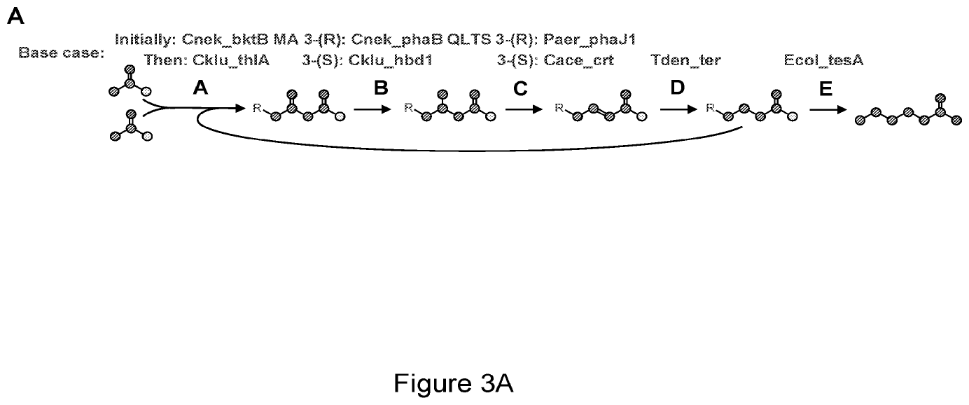

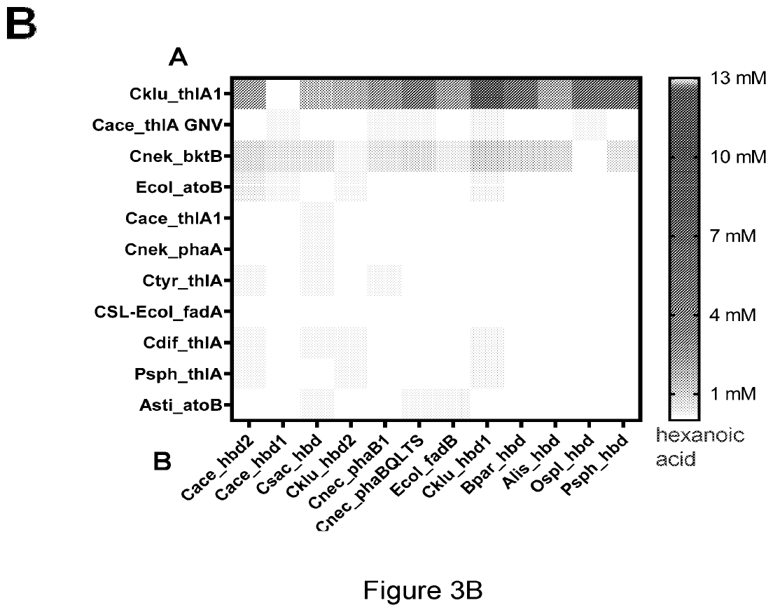

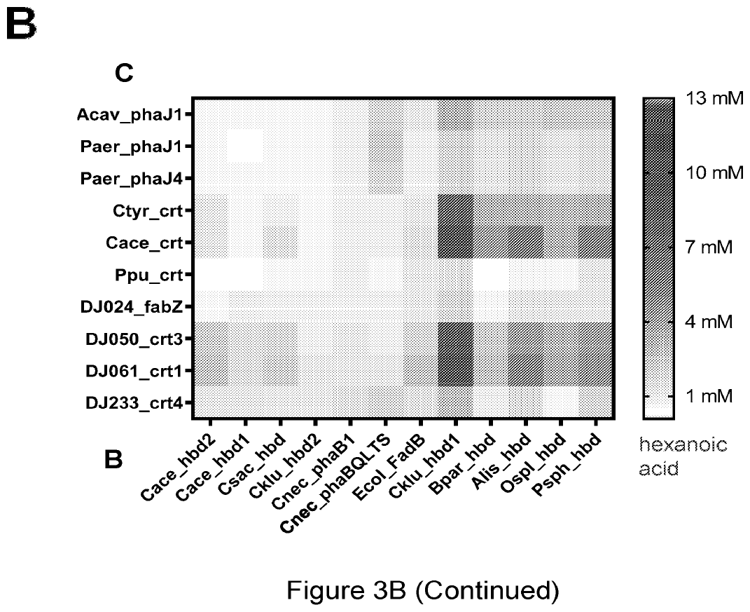

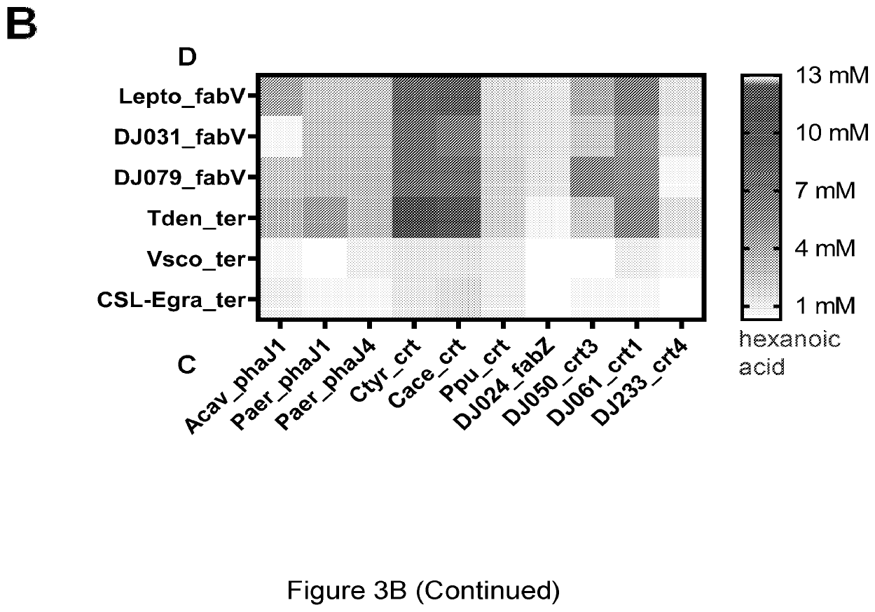

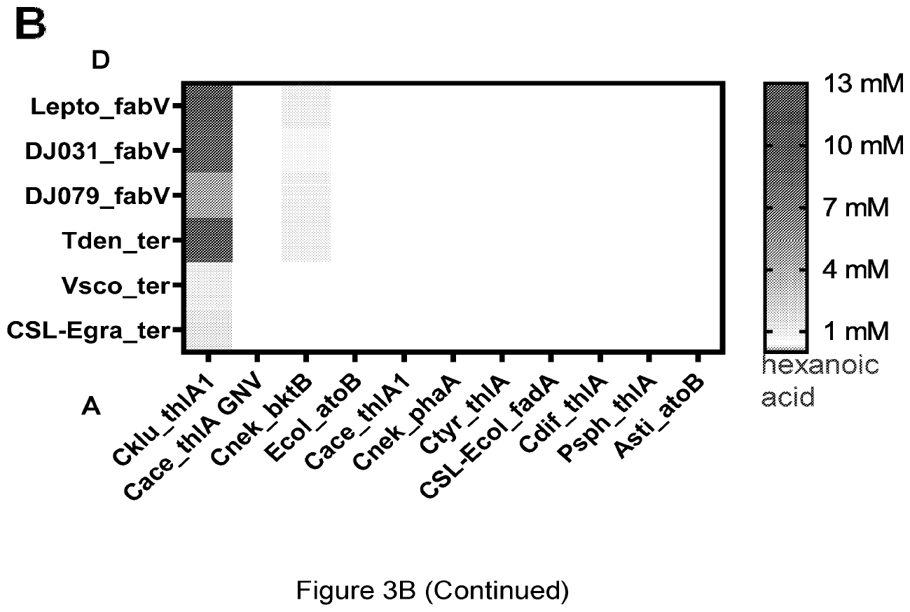

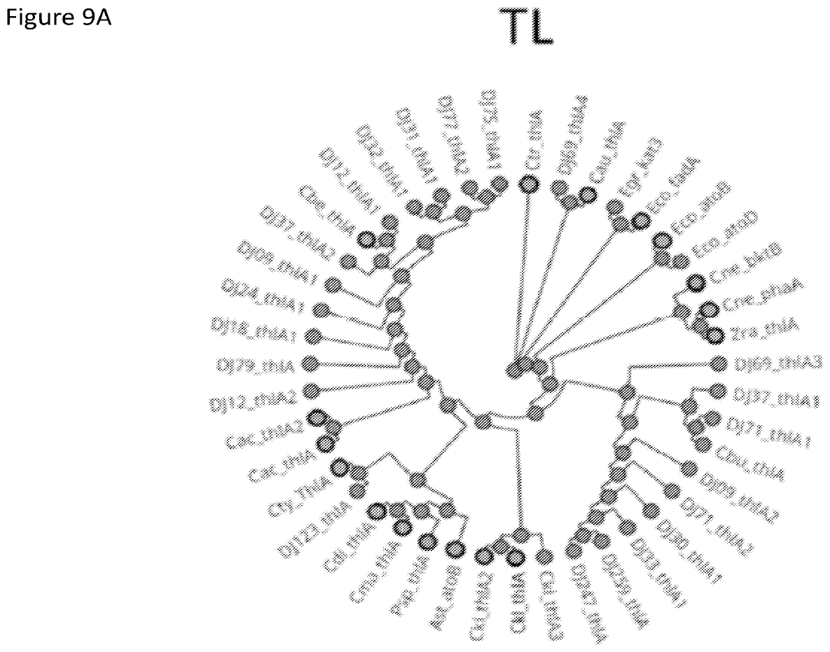

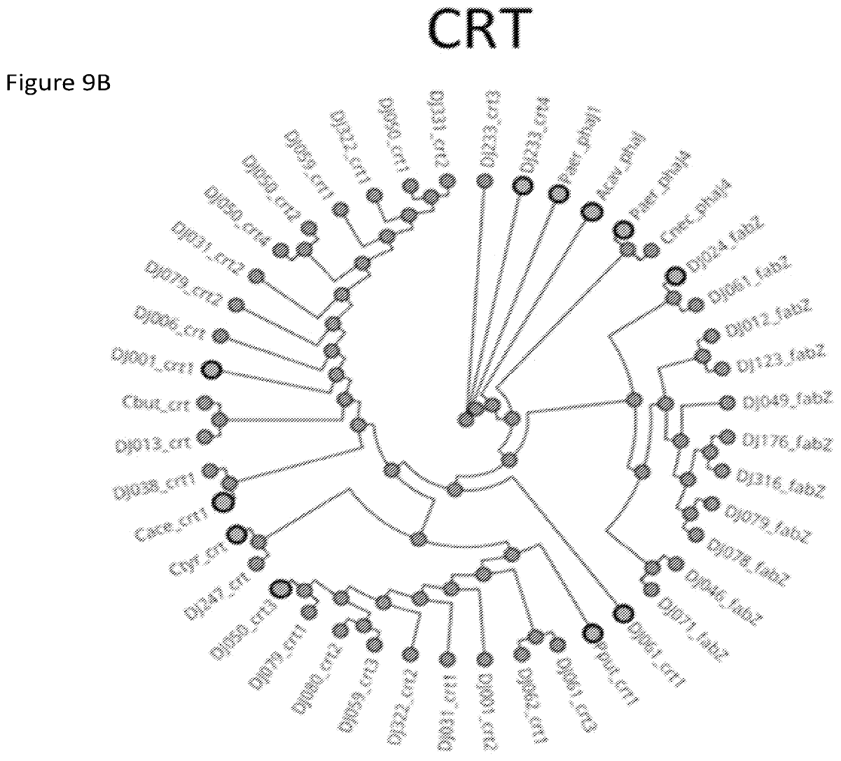

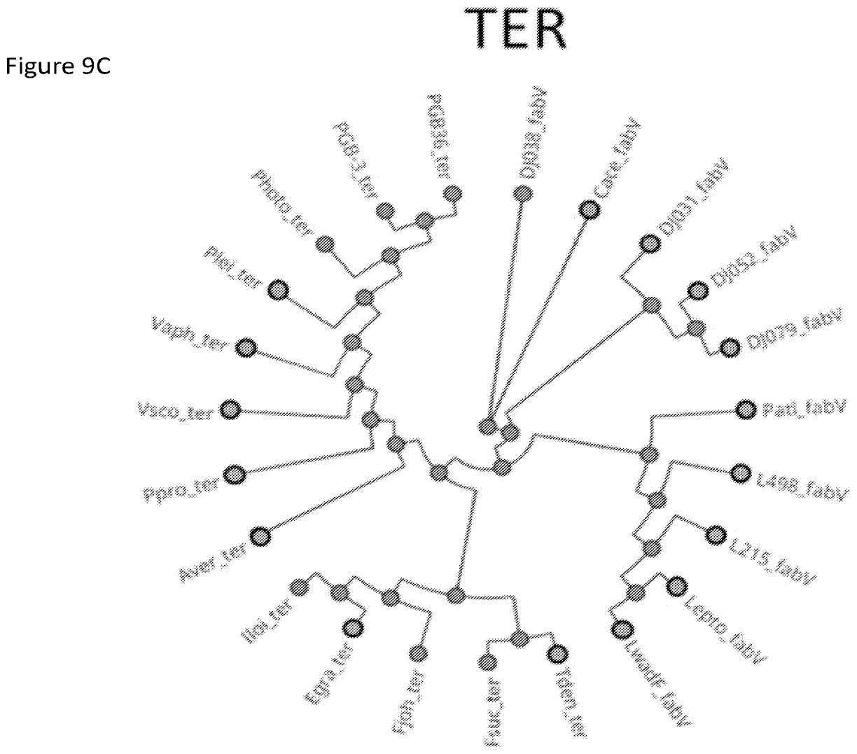

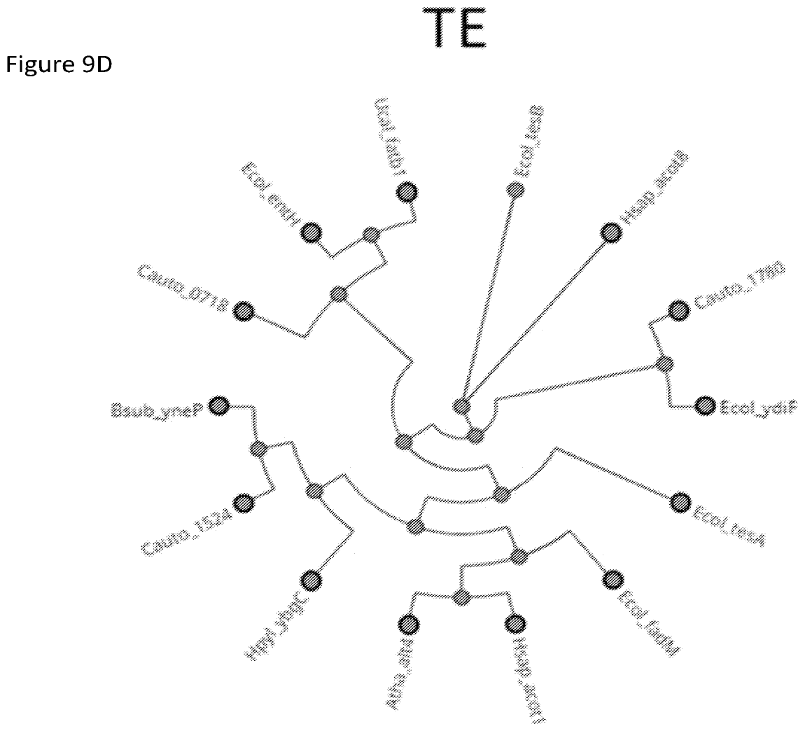

With a functional plate-based screen at hand we mined the sequences from the largest industrial collection of ABE fermentation strains, the David Jones Collection, for r-BOX variants and expanded that selection with a diverse set of enzyme homologues found through literature research and general BLAST analysis. We then picked a diverse set of enzyme homologues for the four enzymes involved in initiation and elongation reactions of r-BOX (TL, HBD, CRT, TER, FIGS. 2C and 9A-D) to be synthesized and tested their soluble expression in our selected r-BOX CFPS extract JST07. Homologues expressing above our cut off of 1 mM in CFPS were then used in a combinatorial screen varying two of the four enzymes while keeping the other two constant. This resulted in a screen of 375 unique enzyme combinations. We first screened all TLs (A) in combination with all HBDs (B) (FIG. 3B, Panel 1). From these 132 different enzyme variants we identified the combination of Cklu_thlA and Cklu_hbd to work more than 5× better than the optimized base case using Cnek_bktB MA (FIG. 3B, Panel 1). We therefore used Cklu_thlA as the new base case TL for the remaining panels. By screening over 375 unique enzyme combinations we identified a pathway that produces 15.7±1.2 mM hexanoic acid. This combination was picked as the starting point for the synthesis of olivetolic acid in the next section.

2. Coupling Optimized Hexanoic Acid Producing r-BOX Variant to Cannabinoid Pathway
2.1 Synthesis of Olivetolic Acid from Glucose

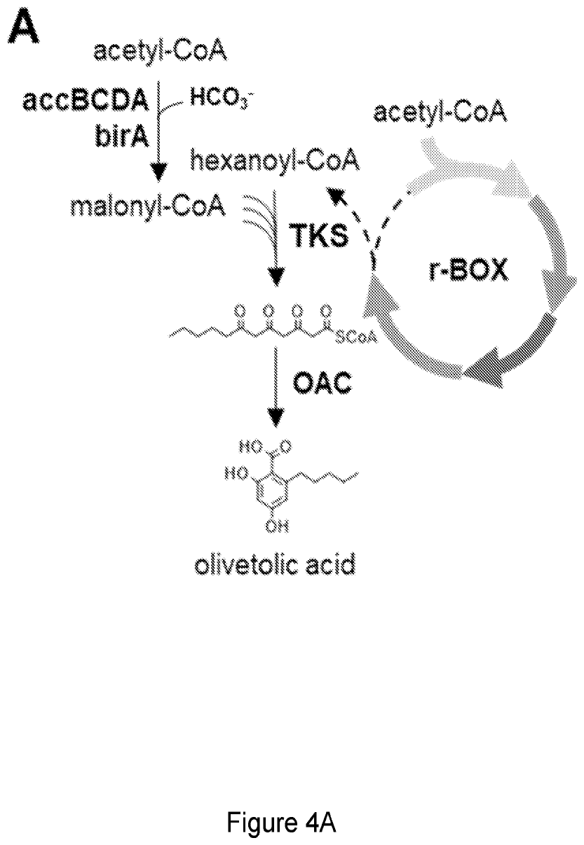

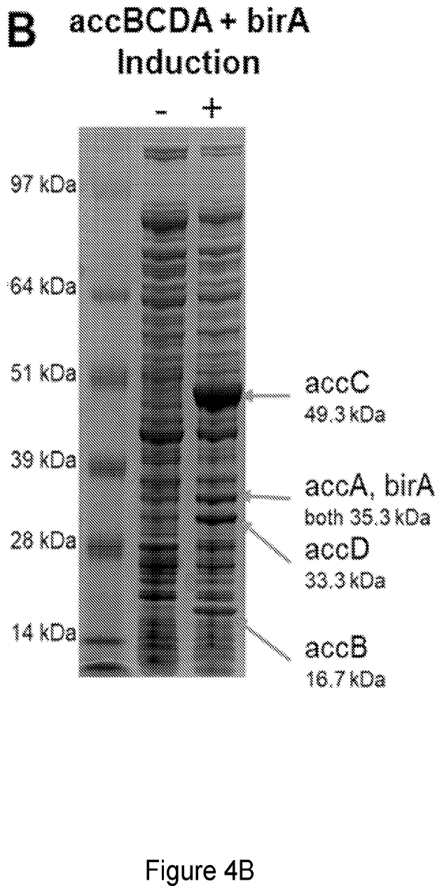

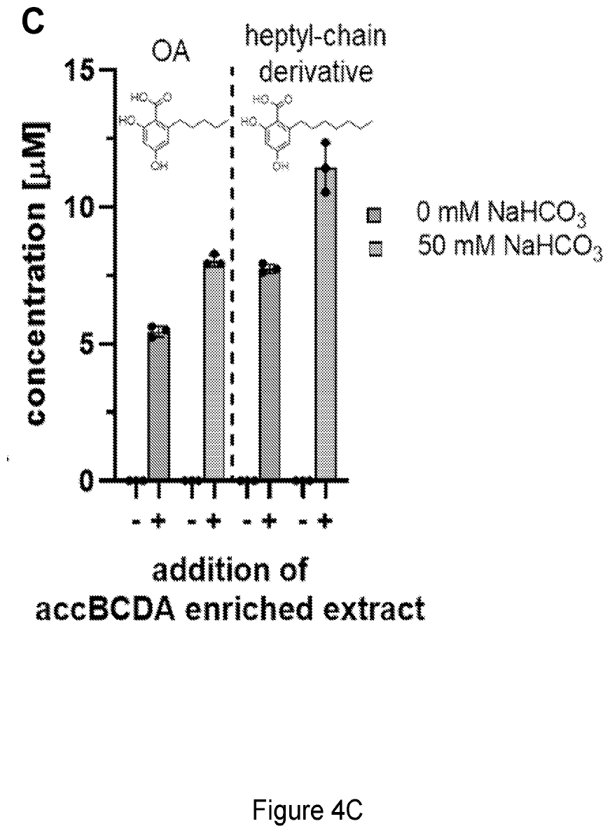

Our optimized r-BOX platform synthesizes high titers of hexanoyl-CoA, the precursor for the synthesis of olivetolic acid (OA), an intermediate in the production of cannabinoids. Starting from r-BOX-derived hexanoyl-CoA, three iterative, decarboxylative Claisen condensations catalyzed by tetraketide synthase (TKS) produce a tetraketide intermediate that is subsequently cyclized by olivetolic acid cyclase (OAC) to form olivetolic acid (FIG. 4A).

Based on pilot experiments that failed to produce OA (data not shown) and previous reports that describe the positive effects of acetyl-CoA carboxylase (acc) overexpression on in vivo OA titers (see Tan, Z. G., J. M. Clomburg, and R. Gonzalez, Synthetic Pathway for the Production of Olivetolic Acid in Escherichia coli. Acs Synthetic Biology, 2018. 7(8): p. 1886-1896), we generated lysates enriched in Acc. Acc was produced in vivo as opposed to in vitro, due to the multi-subunit nature of the protein complex and the required biotinylation. Genes encoding all Acc subunits (accBCDA) and biotin ligase birA (both from E. coli) were overexpressed from a single operon in E. coli JST07, prior to lysate preparation (FIG. 4B). Only upon the addition of accBCDA-enriched extract to fully assembled CFME reactions, were olivetolic acid and the octanoyl-CoA-derived heptyl-chain derivative (2,4-dihydroxy-6-heptylbenzoic acid) produced. The addition of 50 mM sodium bicarbonate further increased product titers to −8 mM (FIG. 4C, the used calibration curve is based on the extinction of OA at 300 nm, but we expect no significant change in e_{300 nm} for the heptyl-chain derivative).

### 2.2 High-Throughput Optimization of Olivetolic Acid Production Platform

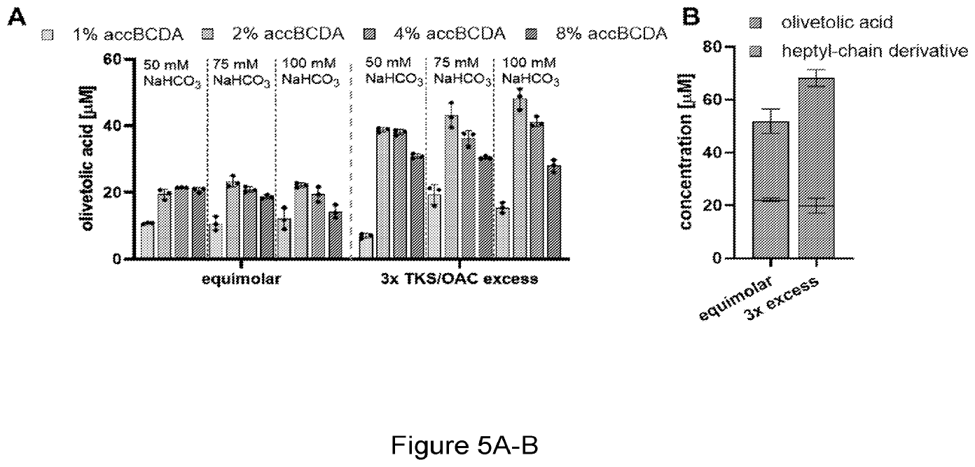

The high concentration of heptyl-chain derivative produced in 2.1, which likely arises from an ‘over-extension’ of the growing acyl-CoA chain in r-BOX (i.e. undesired octanoyl-CoA formation), suggests that removal of hexanoyl-CoA from the r-BOX cycle is not catalyzed efficiently enough to sustain OA-specific product formation. To facilitate increased removal of hexanoyl-CoA from r-BOX, we added both TKS and OAC in three-fold excess relative to all r-BOX enzymes (600 nM vs 200 nM, respectively). Simultaneously, we quantified the effect that increasing NaHCO₃ as well as varying accBCDA-enriched lysate concentrations had on OA titers (FIG. 5A).

Adding a three-fold excess of OA-synthesizing enzymes improved OA titers roughly two-fold, compared to the equimolar control. Addition of 2% v/v accBCDA-enriched extract was found to be optimal and only for this concentration did OA titers linearly increase with increasing NaHCG₃ concentrations. The overall best condition produced roughly 45 mM OA, an approximately five-fold increase over the initial condition (FIG. 5A). Note that, because accBCDA is produced in vivo the concentration cannot be assessed properly, which makes a v/v % optimization necessary for each new accBCDA-lysate batch. While the shift in relative enzyme ratios did not reduce the amount of side products, the overall ratio of OA to off-target product is improved (FIG. 5B).

### 2.3 Cerulenin Addition Increases OA Yield Drastically

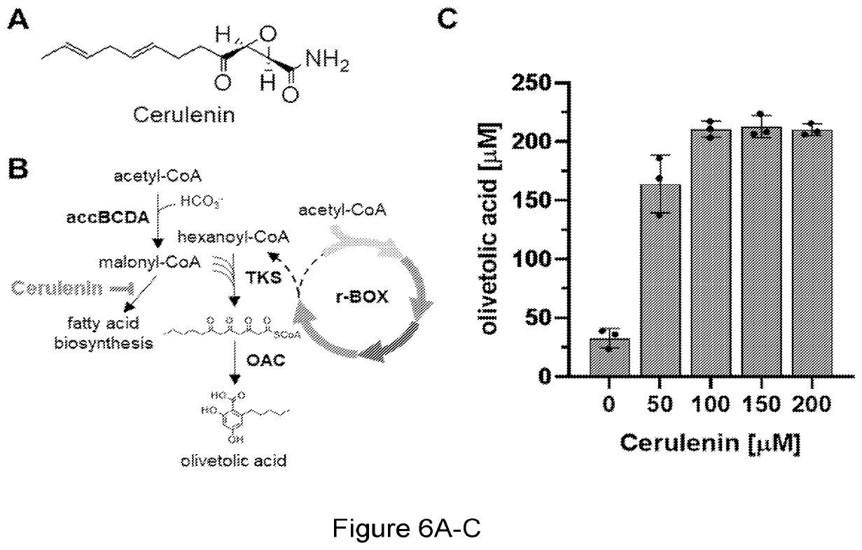

As the continued production of heptyl-chain derivative indicates that hexanoyl-CoA supply by r-BOX is still not production-limiting, we sought to increase the malonyl-CoA pool size. To do so, we pre-incubated CFME reactions with the fatty acid biosynthesis-inhibiting antibiotic cerulenin (FIG. 6A). Cerulenin irreversibly inhibits the b-keto-acyl-ACP synthase of natural fatty acid biosynthesis and thereby minimizes undesired depletion of malonyl-CoA (FIG. 6B) (see Omura, S., Antibiotic Cerulenin, a Novel Tool for Biochemistry as an Inhibitor of Fatty-Acid Synthesis. Bacteriological Reviews, 1976. 40(3): p. 681-697).

The addition of cerulenin improves OA titers from −40 mM to >200 mM and completely abolishes the production of the heptyl-chain derivative (FIG. 6C). To put the newly achieved titers into perspective, at ˜49 mg/L olivetolic acid (215 mM) we currently produce roughly 15× higher titers than comparable, engineered yeasts (see Luo, X. Z., et al., Complete biosynthesis of cannabinoids and their unnatural analogues in yeast. Nature, 2019. 567(7746): p. 123-+) and ˜5× higher titers than engineered E. coli strains (see Tan, Z. G., J. M. Clomburg, and R. Gonzalez, supra), if the biosynthesis is initiated from a single carbon source. E. coli fermentations that overexpress fatty acid importers and grow in the presence of hexanoic acid produce ˜80 mg/L olivetolic acid (see Tan, Z. G., J. M. Clomburg, and R. Gonzalez, supra). Note that cerulenin is unable to inhibit our reverse b-oxidation enzymes, which makes it the optimal probe for this hybrid approach to olivetolic acid biosynthesis.

### 2.4 Production of Cannabigerolic Acid Using Our in Vitro Platform

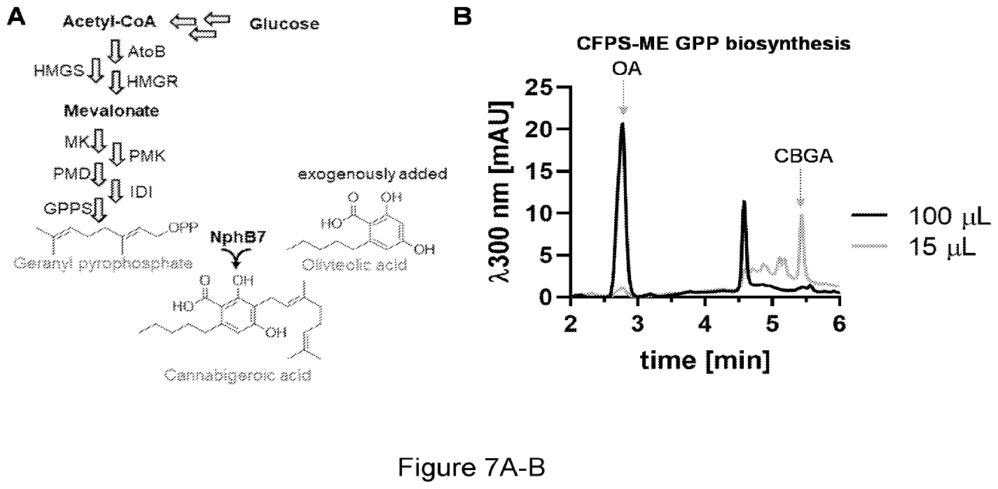

Cannabigerolic acid (CBGA), the penultimate intermediate in the biosynthesis of many phytocannabinoids, is produced by transferring a geranyl moiety to the C3 carbon of olivetolic acid. This transfer is catalyzed by the membrane-bound prenyltransferase cannabigerolic acid synthase (CBGAS). Recently Valliere et al. developed a rationally engineered, soluble enzyme (NphB7) that transfers prenyl moieties to diverse aromatic natural products, including olivetolic acid (see Valliere, M. A., et al., supra). For the synthesis of CBGA we combine the optimized OA-producing assay with previously published enzyme combinations for the in vitro production of geranyl pyrophosphate (GPP) (see Dudley, Q. M., C. J. Nash, and M. C. Jewett, Cell-free biosynthesis of limonene using enzyme-enriched Escherichia coli lysates. Synthetic Biology, 2019. 4(1)) in presence of NphB7 (FIG. 7A). We produced MK, PMK, PMD, IDI and GPPS in vitro using cell-free protein synthesis (CFE) and combined these reactions with HMGR-, HMGS- and AtoB-enriched extract as well as NphB7. The 15 mL reactions were able to convert 0.5 mM OA to CBGA and several unidentified side-products (FIG. 7B, green chromatogram).

The production of CBGAS from olivetolic acid and glucose shows the successful production of GPP, as well as activity of the engineered prenyltransferase NphB7, in vitro. This serves as a great starting point for downstream combination of both the olivetolic acid-synthesizing branch and the GPP-synthesizing branch of the metabolic pathway for the production of CBGAS. Successful combination of the two would facilitate the one-pot synthesis of CBGAS from readily available carbon and energy sources like glucose (one-pot pot synthesis).

TABLE 1

| Abbreviation | Full enzyme name | Function | Source organism | Ref. |
|---|---|---|---|---|
| Abel hbd | 3 hydroxyacyl CoA | Ketoreductase | Azotobacter beijernicki | — |
| | dehydrogenase | (B) | DSM1041 | |
| Acav phal | (R) specific enoyl CoA | Dehydratase (c) | Acromonas cavide | — |
| | hydratase | | | |
| Adn | Fatty acyl CoA reductase | Termination (E) | Adinetobacter baylyi | — |
| Alls hbd | 3 hydroxyacyl CoA | Ketoreductase | Alistipes sp. CAG: 831 | — |
| | dehydrogenase | (B) | | |
| Alvr bbe | Berberine bridge like | Cannabinold | Arabidopsis lyrate | — |
| | enzyme | | | |
| Astil atoB | Acetyl COA | Thiolase (A) | Acetoanzeroblum | — |
| | acetyltransferase | | sticklandil | |
| Asti hbd | 3 hydroxyacyl CoA | Ketoreductase | Acetoanzeroblum | — |
| | dehydrogenase | (B) | sticklandil | |
| Atha alt4 | Acyl ACP thicesterase | Termination (E) | Arabidopsis thaliana | — |
| | ALT4 | | | |
| Atha bbe | Berberine bridge like | Cannabinold | Arabidopsis thaliana | — |
| | enzyme | | | |
| Aver ter | Trans 2 enoyl CoA | Enoyl CoA red. | Aeromonas veronil | — |
| | reductase (NADH) | (D) | | |
| Bpar hbd | 3 hydroxyacyl CoA | Ketoreductase | Butyricimonas paravirosa | — |
| | dehydrogenase | (B) | | |
| Brap bbe | Berberine bridge like | Cannabinold | Brassica rapa | — |
| | enzyme | | | |
| Boub yneP | Putative acyl CoA | Termination (E) | Bacillus subtills | — |
| | thioesterase | | | |
| Cace adhE2 | Alcohol dehydrogenase | Termination (E) | C. acetobutylicum | — |
| | (bifunctional) | | | |
| Cace bed | Butyryl COA | Enoyl CoA red. | C. acetobutylicum | — |
| | dehydrogenase | (D) | | |
| Cace art | Short chain enoyl CoA | Dehydratase (c) | C. acetobutylicum | — |
| | hydratase | | ATCC824 | |
| Cace fabv | Trans 2 enoyl CoA | Enoyl CoA red. | C. acetobutylicum | — |
| | reductase (NADH] | (D) | ATCC824 | |
| Cace hbdl | 3 CoA dehydrogenase | Ketoreductase | C. acetobutylicum | — |
| | | (B) | ATCC824 | |
| Cace hbd2 | 3 CoA dehydrogenase | Ketoreductase | C. acetobutylicum | — |
| | | (B) | ATCC824 | |
| Cace thia | Acetyl CoA | Thiolase (A) | C. acetobutylicum | — |
| | acetyltransferase | | ATCC824 | |
| Caut thIa | Acetyl CoA | Thiolase (A) | C. autoethanogenum | — |
| | acetyltransferase | | | |
| Cauto 0718 | Palmitoyl COA hydrolase | Termination (E) | C. autoethanogenum | — |
| Cauto 1524 | CoA thioesterase | Termination (E) | C. autoethanogenum | — |
| Cauto 1780 | Uncharacterized protein | Termination (E) | C. autoethanogenum | — |
| CBDAS | Cannabidiolic acid | Cannabinold | Cannabis sativa | — |
| | synthase | | | |
| Cbel adh | Alcohol dehydrogenase | Termination (E) | C. beijerinickil | — |
| | (bifunctional) | | | |
| Cbel hbd | CoA dehydrogenase | Ketoreductase | C. beijerinckii | — |
| | | (B) | | |
| Cbel ptb | Phosphate | Termination (E) | C. beijerinickii | — |
| | butyryltransferase | | | |
| Cbel thiA | Aceityl COA | Thiolase (A) | C. beijerinckil | — |
| | acetyltransferase | | | |
| CBGAS | Cannabigerolic acid | Cannabinold | Cannabis sativa | — |
| | synthase | | | |
| Cbut hbd | 3 COA dehydrogenase | Ketoreductase | C. butyricum | — |
| | | (B) | | |
| Cbut ptb | Phosphate | Termination (E) | C. butyricum | — |
| | butyryltransferase | | | |
| Cdif hbd | CoA dehydrogenase | Ketoreductase | C. difficile CD10-165 | — |
| | | (B) | | |
| Cdif thia | Acetyl CoA | Thiolase (A) | C. difficile CD10-165 | — |
| | acentyltransferase | | | |
| Cidu hbdl | 3 dehydrogenase | Ketoreductase | C. kluyveri | — |
| | | (B) | | |
| Cklu hbd2 | 3 hydroxyacyl CoA | Ketoreductase | C. kluyveri | — |
| | dehydrogenase | (B) | | |
| | Acetyl COA | Thiolase (A) | C. kluyveri | — |
| | acetyltransferase | | | |
| Cklu thiaz | Acetyl COA | Thiolase (A) | C. kluyveri | — |
| | acetyltransferase | | | |
| Cidu thiA3 | Acetyl COA | Thiolase (A) | C. kluyveri | — |
| | acctyltranderass | | | |
| Cman hbd | 3 COA dehydrogenase | Ketoreductase | C. mangenotili TR | — |
| | | (B) | | |
| Cman thiA | Aceityl COA | Thiolase (A) | C. mangenotil TR | — |
| | acetyltransferase | | | |
| Csac hbd | 3 hydroxyacyl CoA | Ketoreductase | C. saccharoperbutylacetonicum | — |
| | dehydrogenase | (B) | | |
| Ctro thia | Aceityl CoA | Thiolase (A) | Candida tropicalis | — |
| Ctyr bed | Butyryl CoA | Enoyl CoA red. | C. tyrobutyricum | — |
| | dehydrogenase | (D) | | |
| Ctyr crt | Short chain enoyl COA | Dehydratase (c) | C. tyrobutyricum | — |
| | hydratase | | | |
| Ctyr hbd | 3 hydroxacyl COA | Ketoreductase | C. tyrobuturicum | — |
| | dehydrogenase | (B) | | |
| Ctyr thiA | Acetyl COA | Thiolase (A) | C. tyrobutyricum | — |
| DJ001 art2 | Short chain enoyl CoA | Dehydratase (C) | DJ collection (001) | — |
| | hydratase | | | |
| DJ006 acr1 | Fatty acyl CoA reductase | Termination (E) | DJ collection (006) | — |
| DJ008 acr1 | Fatty acyl CoA reductase | Termination | DJ collection (008) | — |
| DJ024 fabz | Short chainenoyl COA | Dehydratase (c) | DJ collection (024) | — |
| | hydratase | | | |
| DJ024 ptb | Phosphate | Termination (E) | DJ collection (024) | — |
| | butyryltransferase | | | |
| DJOSO crt3 | Short chain enoyl CoA | Dehydratase | DJ collection (050) | — |
| | hydratase | | | |
| DJ052 acr1 | Fatty acyl CoA reductase | Termination (E) | DJ collection (052) | — |
| DJ052 buk | Butyryl kinase | Termination (E) | DJ collection (052) | — |
| DJ052 fabV | Trans 2 enoyl CoA | Enoyl CoA red. | DJ collection (052) | — |
| | reductase \|NADH] | (D) | | |
| DJ061 crtl | Short chain enoyl CoA | Dehydratase (c) | DJ collection (061) | — |
| | hydratase | | | |
| DJ062 buk | Butyryl kinase Phosphate | Termination (E) | DJ collection (062) | — |
| DJ062 ptb | butyryltransferase | Termination (E) | DJ collection (062) | — |
| DJ079_acr1 | Fatty acyl-CoA reductase | Termination (E) | DJ collection (079) | — |
| DJ079_fabV | Trans-2-enoyl-CoA | Enoyl-CoA red. | DJ collection (079) | — |
| | reductase [NADH] | (D) | | |
| DJ233_crt4 | Short-chain-enoyl-CoA | Dehydratase (C) | DJ collection (233) | — |
| | hydratase | | | |
| DJ322_acr1 | Fatty acyl-CoA reductase | Termination (E) | DJ collection (322) | — |
| Ecol_ackA | Acetate kinase | Miscellaneous | Escherichia coli | — |
| Ecol_atoB | Acetyl-CoA | Thiolase (A) | Escherichia coli | — |
| | acetyltransferase | | | |
| Ecol_entH | Proofreading thioesterase | Termination (E) | Escherichia coli | — |
| Ecol_fadA | 3-ketoacyl-CoA thiolase | Thiolase (A) | Escherichia coli | — |
| Ecol_fadB | Fatty acid oxidation | Ketoreductase | Escherichia coli | — |
| | complex subunit a | (B) | | |
| Ecol_fadM | Long-chain acyl-CoA | Termination (E) | Escherichia coli | — |
| | thioesterase | | | |
| Ecol_pta | Phosphotransacetylase | Miscellaneous | Escherichia coli | — |
| Ecol_tesA | Thioesterase 1 | Termination (E) | Escherichia coli | — |
| Egra_ter | Trans-2-enoyl-CoA | Enoyl-CoA red. | Euglena gracilis | — |
| | reductase [NADH] | (D) | | |
| GPPS | Geranyl pyrophosphate | GPP biosynthesis | Abies grandis | — |
| | synthase | | | |
| HMGR | HMG-CoA reductase | GPP biosynthesis | Pseudomonas. mevalonii | — |
| HMGS | HMG-CoA synthase | GPP biosynthesis | Saccharomyces cerevisiae | — |
| Hpyl_ybgC | Acyl-CoA thioester | Termination (E) | Helicobacter pylori | — |
| | hydrolase | | | |
| Hsap_acot13 | Acyl-coenzyme A | Termination (E) | Homo sapiens | — |
| | thioesterase 13 | | | |
| Hsap_acot8 | Acyl-coenzyme A | Termination (E) | Homo sapiens | — |
| | thioesterase 8 | | | |
| IDI | Isopentenyl pyrophosphate | GPP biosynthesis | Escherichia coli | — |
| | isomerase | | | |
| L215_fabV | Trans-2-enoyl-CoA | Enoyl-CoA red. | Leptotrichia sp. OT215 | — |
| | reductase [NADH] | (D) | | |
| Lepto_fabV | Trans-2-enoyl-CoA | Enoyl-CoA red. | Leptotrichia sp. | — |
| | reductase [NADH] | (D) | | |
| LwadF_fabV | Trans-2-enoyl-CoA | Enoyl-CoA red. | Leptotrichia wadei | — |
| | reductase [NADH] | (D) | | |
| Mann_thlA | Acetyl-CoA | Thiolase (A) | C. acetobutylicum | — |
| | acetyltransferase | | ATCC824 | |
| Maqu2507 | Fatty acyl-CoA reductase | Termination (E) | Marinobacter aquaeolei | — |
| MK | Mevalonate kinase | GPP biosynthesis | Saccharomyces. | — |
| | | | cerevisiae | |
| Mmaz_mk | Mevalonate kinase | GPP biosynthesis | Methanosarcina mazei | — |
| Natt_bbe | Berberine-bridge like | Cannabinoid | Nicotiana attenuate | — |
| | enzyme | | | |
| NphB7 | Engineered | Cannabinoid | Cannabis sativa | — |
| | prenyltransferase | | | |
| OAC | Olivetolic acid cyclase | Cannabinoid | Cannabis sativa | — |
| Ospl_hbd | 3-hydroxyacyl-CoA | Ketoreductase | Odoribacter splanchnicus | — |
| | dehydrogenase | (B) | | |
| Paer_phaJ1 | (R)-specific enoyl-CoA | Dehydratase (C) | Pseudomonas aeruginosa | — |
| | hydratase | | | |
| Paer_phaJ4 | (R)-specific enoyl-CoA | Dehydratase (C) | Pseudomonas aeruginosa | — |
| | hydratase | | | |
| Pgla_gpps | Geranyl pyrophosphate | GPP biosynthesis | Picea glauca | — |
| | synthase | | | |
| Plei_ter | Trans-2-enoyl-CoA | Enoyl-CoA red. | Photobacterium | — |
| | reductase [NADH] | (D) | leiognathi | |
| PMD | Pyrophosphomevalonate | GPP biosynthesis | Saccharomyces cerevisiae | — |
| | decarboxylase | | | |
| PMK | Phophomevalonate kinase | GPP biosynthesis | Saccharomyces cerevisiae | — |
| Pper_bbe | Berberine-bridge like | Cannabinoid | Prunus persica | — |
| | enzyme | | | |
| Ppro_ter | Trans-2-enoyl-CoA | Enoyl-CoA red. | Photobacterium | — |
| | reductase [NADH] | (D) | profundum | |
| Ppsh_hbd | 3-hydroxyacyl-CoA | Ketoreductase | Proteocatella sphenisci | — |
| | dehydrogenase | (B) | | |
| Pput_crt1 | Short-chain-enoyl-CoA | Dehydratase (C) | Pseudomonas putida | — |
| | hydratase | | | |
| Psom_bbe | Berberine-bridge like | Cannabinoid | Papaver somniferum | — |
| | enzyme | | | |
| Psph_thlA | Acetyl-CoA | Thiolase (A) | Proteocatella sphenisci | — |
| | acetyltransferase | | | |
| Pzea_pmk | Phophomevalonate kinase | GPP biosynthesis | Paracoccus | — |
| | | | zeaxanthinifaciens | |
| Reut_bktB | Beta-ketothiolase M158A | Thiolase (A) | Ralstonia eutropha | https://doi.org/10.1002/bit.26737 |
| Reut_phaA | Acetyl-CoA | Thiolase (A) | Ralstonia eutropha | — |
| | acetyltransferase | | | |
| Reut_phaB | Acetoacetyl-CoA reductase | Ketoreductase | Ralstonia eutropha | — |
| | | (B) | | |
| Reut_phaB | Acetoacetyl-CoA reductase | Ketoreductase | Ralstonia eutropha | https://doi.org/10.1128/AEM.01768-13 |
| QLTS | Q47L T173S | (B) | | |
| Saur_idi | Isopentenyl pyrophosphate | GPP biosynthesis | Staphlyococcus aureus | — |
| | isomerase | | | |
| Scer_pmd | Pyrophosphomevalonate | GPP biosynthesis | Saccharomyces cerevisiae | — |
| | decarboxylase | | | |
| Stre_nphT7 | Acetoacetyl-CoA synthase | Thiolase (A) | Streptomyces sp. | — |
| | | | (strain CL190) | |
| Tden_ter | Trans-2-enoyl-CoA | Enoyl-CoA red. | Treponema denticola | — |
| | reductase [NADH] | (D) | | |
| THCAS | Tetrahydrocannabinol | Cannabinoid | Cannabis sativa | — |
| | synthase | | | |
| TKS | Tetraketide synthase | Cannabinoid | Cannabis sativa | — |
| Ucal_fatb1 | Dodecanoyl-ACP | Termination (E) | Umbellularia californica | — |
| | hydrolase | | | |
| Vaph_ter | Trans-2-enoyl-CoA | Enoyl-CoA red. | Vibrio aphrogenes | — |
| | reductase [NADH] | (D) | | |
| Vsco_ter | Trans-2-enoyl-CoA | Enoyl-CoA red. | Vibrio scophtalmi | — |
| | reductase [NADH] | (D) | | |
| Zram_phaA | Acetyl-CoA | Thiolase (A) | Zoogloea ramigera | — |
| | acetyltransferase | | | |

TABLE 2

| | | | Total | Soluble | Extract |
|---|---|---|---|---|---|
| Abbreviation | Function | Source organism | [mM] | [mM] | # |
| Abei_hbd | Ketoreductase (B) | Azotobacter beijernicki | 0.69 ± 0.04 | 0.64 ± 0.06 | JST07-5 |
| | | DSM1041 | | | |
| Acav_phaJ | Dehydratase (C) | Aeromonas caviae | — | 19.72 ± 2.89 | JST07-5 |
| Acin_acr1 | Termination (E) | Acinetobacter baylyi | 1.51 ± 0.73 | 0.81 ± 0.34 | JST07-4 |
| Alis_hbd | Ketoreductase (B) | Alistipes sp. CAG: 831 | 15.32 ± 2.55 | 14.2 ± 3.36 | JST07-5 |
| Alyr_bbe | Cannabinoid | Arabidopsis lyrate | — | — | — |
| Asti_atoB | Thiolase (A) | Acetoanaerobium | — | 1.24 ± 0.11 | JST07-5 |
| | | sticklandii | | | |
| Asti_hbd | Ketoreductase (B) | Acetoanaerobium | — | — | — |
| | | sticklandii | | | |
| Atha_alt4 | Termination (E) | Arabidopsis thaliana | — | 2.58 ± 0.19 | JST07-8 |
| Atha_bbe | Cannabinoid | Arabidopsis thaliana | — | — | JST07-5 |
| Aver_ter | Enoyl-CoA red. | Aeromonas veronii | 2.56 ± 0.41 | 1.23 ± 0.38 | JST07-4 |
| | (D) | | | | |
| Bpar_hbd | Ketoreductase (B) | Butyricimonas paravirosa | — | 12.01 ± 1.57 | JST07-5 |
| Brap_bbe | Cannabinoid | Brassica rapa | — | — | — |
| Bsub_yneP | Termination (E) | Bacillus subtilis | 5.56 ± 1.14 | 4.89 ± 2.22 | JST07-5 |
| Cace_adhE2 | Termination (E) | C. acetobutylicum | — | 1.41 ± 0.49 | JST07-5 |
| Cace_bcd | Enoyl-CoA red. | C. acetobutylicum | — | — | — |
| | (D) | | | | |
| Cace_crt | Dehydratase (C) | C. acetobutylicum | 22.21 ± 0.49 | 23.44 ± 1.88 | JST07-4 |
| | | ATCC824 | | | |
| Cace_fabV | Enoyl-CoA red. | C. acetobutylicum | 4.89 ± 0.42 | 0.64 ± 0.06 | JST07-4 |
| | (D) | ATCC824 | | | |
| Cace_hbd1 | Ketoreductase (B) | C. acetobutylicum | 30.24 ± 7.77 | 4.13 ± 0.45 | JST07-4 |
| | | ATCC824 | | | |
| Cace_hbd2 | Ketoreductase (B) | C. acetobutylicum | 8.18 ± 1.47 | 4.06 ± 1.55 | JST07-4 |
| | | ATCC824 | | | |
| Cace_thlA | Thiolase (A) | C. acetobutylicum | 9.16 ± 0.38 | 8.15 ± 0.74 | JST07-4 |
| | | ATCC824 | | | |
| Caut_thlA | Thiolase (A) | C. autoethanogenum | 4.10 ± 0.35 | 0.54 ± 0.05 | JST07-4 |
| Cauto_0718 | Termination (E) | C. autoethanogenum | 14.15 ± 1.10 | 8.99 ± 0.32 | JST07-4 |
| Cauto_1524 | Termination (E) | C. autoethanogenum | 60.49 ± 15.5 | 8.26 ± 0.9 | JST07-4 |
| Cauto_1780 | Termination (E) | C. autoethanogenum | 61.79 ± 4.31 | 56.74 ± 1.81 | JST07-4 |
| CBDAS | Cannabinoid | Cannabis sativa | 6.26 ± 0.82 | 1.83 ± 0.44 | JST07-5 |
| Cbei_adh | Termination (E) | C. beijerinickii | 1.28 ± 0.20 | 0.97 ± 0.15 | JST07-4 |
| Cbei_hbd | Ketoreductase (B) | C. beijerinckii | — | — | — |
| Cbei_ptb | Termination (E) | C. beijerinickii | 3.85 ± 0.49 | 3.66 ± 0.28 | JST07-4 |
| Cbei_thlA | Thiolase (A) | C. beijerinckii | 1.99 ± 0.35 | 1.18 ± 0.20 | JST07-5 |
| CBGAS | Cannabinoid | Cannabis sativa | — | — | — |
| Cbut_hbd | Ketoreductase (B) | C. butyricum | 1.13 ± 0.23 | 0.56 ± 0.09 | JST07-4 |
| Cbut_ptb | Termination (E) | C. butyricum | 1.62 ± 0.12 | 1.05 ± 0.25 | JST07-4 |
| Cdif_hbd | Ketoreductase (B) | C. difficile CD10-165 | — | — | — |
| Cdif_thlA | Thiolase (A) | C. difficile CD10-165 | — | 0.74 ± 0.09 | JST07-8 |
| Cklu_hbd1 | Ketoreductase (B) | C. kluyveri | 5.78 ± 0.21 | 5.48 ± 0.49 | JST07-4 |
| Cklu_hbd2 | Ketoreductase (B) | C. kluyveri | 13.95 ± 0.97 | 12.81 ± 0.41 | JST07-4 |
| Cklu_th1A1 | Thiolase (A) | C. kluyveri | 5.57 ± 0.72 | 2.58 ± 0.25 | JST07-4 |
| Cklu_th1A2 | Thiolase (A) | C. kluyveri | — | — | — |
| Cklu_th1A3 | Thiolase (A) | C. kluyveri | 3.42 ± 0.37 | 1.54 ± 0.37 | JST07-4 |
| Cman_hbd | Ketoreductase (B) | C. mangenotii TR | — | 0.35 ± 0.18 | JST07-8 |
| Cman_thlA | Thiolase (A) | C. mangenotii TR | — | 0.13 ± 0.07 | JST07-8 |
| Csac_hbd | Ketoreductase (B) | C. | 16.48 ± 2.96 | 8.43 ± 2.94 | JST07-4 |
| | | saccharoperbutylacetonicum | | | |
| Ctro_thlA | Thiolase (A) | Candida tropicalis | 2.87 ± 0.46 | 1.38 ± 0.42 | JST07-4 |
| Ctyr_bcd | Enoyl-CoA red. | C. tyrobutyricum | — | — | — |
| | (D) | | | | |
| Ctyr_crt | Dehydratase (C) | C. tyrobutyricum | 17.75 ± 0.63 | 7.51 ± 3.51 | JST07-4 |
| Ctyr_thlA | Thiolase (A) | C. tyrobutyricum | 2.23 ± 0.31 | 1.63 ± 0.18 | JST07-4 |
| DJ001_crt2 | Dehydratase (C) | DJ collection (001) | — | 1.28 ± 0.33 | JST07-8 |
| DJ006_acr1 | Termination (E) | DJ collection (006) | 1.43 ± 0.18 | 0.46 ± 0.20 | JST07-4 |
| DJ008_acr1 | Termination (E) | DJ collection (008) | 1.76 ± 0.19 | 0.65 ± 0.23 | JST07-4 |
| DJ024_fabZ | Dehydratase (C) | DJ collection (024) | — | 8.72 ± 2.66 | JST07-8 |
| DJ050_crt3 | Dehydratase (C) | DJ collection (050) | — | 3.63 ± 1.86 | JST07-5 |
| DJ052_acr1 | Termination (E) | DJ collection (052) | — | 2.41 ± 0.12 | JST07-5 |
| DJ052_buk | Termination (E) | DJ collection (052) | — | 4.82 ± 0.53 | JST07-5 |
| DJ052_fabV | Enoyl-CoA red. | DJ collection (052) | — | 0.58 ± 0.05 | JST07-5 |
| | (D) | | | | |
| DJ061_crt1 | Dehydratase (C) | DJ collection (061) | — | 5.79 ± 0.23 | JST07-5 |
| DJ062_buk | Termination (E) | DJ collection (062) | — | 2.30 ± 0.31 | JST07-5 |
| DJ079_acr1 | Termination (E) | DJ collection (079) | 0.73 ± 0.11 | 0.08 ± 0.06 | JST07-4 |
| DJ079_fabV | Enoyl-CoA red. | DJ collection (079) | — | 1.94 ± 0.38 | JST07-5 |
| | (D) | | | | |
| DJ233_crt4 | Dehydratase (C) | DJ collection (233) | — | 17.3 ± 3.22 | JST07-5 |
| DJ322_acr1 | Termination (E) | DJ collection (322) | 0.51 ± 0.05 | 0.15 ± 0.05 | JST07-4 |
| Ecol_ackA | Miscellaneous | Escherichia coli | 11.38 ± 2.54 | 11.27 ± 2.99 | JST07-4 |
| Ecol_atoB | Thiolase (A) | Escherichia coli | 14.74 ± 0.39 | 6.70 ± 0.39 | JST07-4 |
| Ecol_entH | Termination (E) | Escherichia coli | — | 17.41 ± 1.63 | JST07-8 |
| Ecol_fadA | Thiolase (A) | Escherichia coli | — | 6.73 ± 1.19 | JST07-8 |
| Ecol_fadB | Ketoreductase (B) | Escherichia coli | — | 2.26 ± 0.87 | JST07-8 |
| Ecol_fadM | Termination (E) | Escherichia coli | — | 5.09 ± 1.30 | JST07-8 |
| Ecol_pta | Miscellaneous | Escherichia coli | — | 3.66 ± 0.11 | JST07-8 |
| Ecol_tesA | Termination (E) | Escherichia coli | 13.86 ± 0.89 | 1.93 ± 0.34 | JST07-4 |
| Egra_ter | Enoyl-CoA red. | Euglena gracilis | — | 3.70 ± 1.04 | JST07-8 |
| | (D) | | | | |
| GPPS | GPP biosynthesis | Abies grandis | — | — | — |
| HMGR | GPP biosynthesis | Pseudomonas. mevalonii | — | — | — |
| HMGS | GPP biosynthesis | Saccharomyces cerevisiae | — | — | — |
| Hpyl_ybgC | Termination (E) | Helicobacter pylori | — | 7.71 ± 1.56 | JST07-8 |
| Hsap_acot13 | Termination (E) | Homo sapiens | — | 13.5 ± 1.29 | JST07-8 |
| Hsap_acot8 | Termination (E) | Homo sapiens | — | 0.33 ± 0.07 | JST07-8 |
| IDI | GPP biosynthesis | Escherichia coli | — | — | — |
| L215_fabV | Enoyl-CoA red. | Leptotrichia sp. OT215 | 1.35 ± 0.17 | 1.06 ± 0.12 | JST07-4 |
| | (D) | | | | |
| Lepto_fabV | Enoyl-CoA red. | Leptotrichia sp. | 14.67 ± 1.47 | 3.57 ± 0.69 | JST07-4 |
| | (D) | | | | |
| LwadF_fabV | Enoyl-CoA red. | Leptotrichia wadei | — | 1.39 ± 0.38 | JST07-5 |
| | (D) | | | | |
| Maqu2507 | Termination (E) | Marinobacter aquaeolei | 1.61 ± 0.4 | 0.30 ± 0.13 | JST07-4 |
| MK | GPP biosynthesis | Saccharomyces. cerevisiae | — | — | JST07-5 |
| Mmaz_mk | GPP biosynthesis | Methanosarcina mazei | | 19.99 ± 8.84 | JST07-11 |
| Natt_bbe | Cannabinoid | Nicotiana attenuate | — | — | — |
| NphB7 | Cannabinoid | Engineered | — | 4.73 ± 0.47 | JST07-10 |
| OAC | Cannabinoid | Cannabis sativa | — | 3.26 ± 0.57 | JST07-10 |
| Ospl_hbd | Ketoreductase (B) | Odoribacter splanchnicus | — | 14.52 ± 4.15 | JST07-8 |
| Paer_phaJ1 | Dehydratase (C) | Pseudomonas aeruginosa | 16.25 ± 2.08 | 13.24 ± 0.14 | JST07-4 |
| Paer_phaJ4 | Dehydratase (C) | Pseudomonas aeruginosa | 22.15 ± 3.00 | 17.98 ± 0.81 | JST07-4 |
| Pgla_gpps | GPP biosynthesis | Picea glauca | — | 9.00 ± 2.51 | JST07-11 |
| Plei_ter | Enoyl-CoA red. | Photobacterium leiognathi | 1.88 ± 0.41 | 0.24 ± 0.06 | JST07-4 |
| | (D) | | | | |
| PMD | GPP biosynthesis | Saccharomyces cerevisiae | — | — | — |
| PMK | GPP biosynthesis | Saccharomyces cerevisiae | — | — | — |
| Pper_bbe | Cannabinoid | Prunus persica | — | — | — |
| Ppro_ter | Enoyl-CoA red. | Photobacterium profundum | 12.14 ± 1.12 | 0.27 ± 0.10 | JST07-4 |
| | (D) | | | | |
| Ppsh_hbd | Ketoreductase (B) | Proteocatella sphenisci | — | 2.24 ± 0.22 | JST07-8 |
| Pput_crt1 | Dehydratase (C) | Pseudomonas putida | — | 15.96 ± 5.37 | JST07-8 |
| Psom_bbe | Cannabinoid | Papaver somniferum | — | — | — |
| Psph_thlA | Thiolase (A) | Proteocatella sphenisci | — | 4.36 ± 0.79 | JST07-8 |
| Pzea_pmk | GPP biosynthesis | Paracoccus | — | 11.71 ± 2.81 | JST07-11 |
| | | zeaxanthinifaciens | | | |
| Reut_bktB | Thiolase (A) | Ralstonia eutropha | 10.43 ± 1.87 | 5.34 ± 1.83 | JST07-4 |
| Reut_phaA | Thiolase (A) | Ralstonia eutropha | 9.69 ± 0.39 | 8.65 ± 0.78 | JST07-4 |
| Reut_phaB | Ketoreductase (B) | Ralstonia eutropha | 18.33 ± 0.75 | 16.3 ± 1.47 | JST07-4 |
| Reut_phaB Q | Ketoreductase (B) | Ralstonia eutropha | 38.34 ± 1.00 | 17.41 ± 1.01 | JST07-4 |
| Saur_idi | GPP biosynthesis | Staphlyococcus aureus | — | 9.30 ± 1.36 | JST07-11 |
| Scer_pmd | GPP biosynthesis | Saccharomyces cerevisiae | — | 4.36 ± 0.75 | JST07-11 |
| Tden_ter | Enoyl-CoA red. | Treponema denticola | 13.16 ± 0.76 | 11.55 ± 0.75 | JST07-4 |
| | (D) | | | | |
| THCAS | Cannabinoid | Cannabis sativa | 4.99 ± 0.37 | 0.85 ± 0.45 | JST07-8 |
| TKS | Cannabinoid | Cannabis sativa | — | 5.02 ± 0.62 | JST07-10 |
| Ucal_fatb1 | Termination (E) | Umbellularia californica | — | 0.60 ± 0.36 | JST07-8 |
| Vaph_ter | Enoyl-CoA red. | Vibrio aphrogenes | — | 0.76 ± 0.27 | JST07-8 |
| | (D) | | | | |
| Vsco_ter | Enoyl-CoA red. | Vibrio scophtalmi | — | 0.82 ± 0.05 | JST07-8 |
| | (D) | | | | |
| Zram_phaA | Thiolase (A) | Zoogloea ramigera | 2.61 ± 0.56 | 0.34 ± 0.09 | JST07-4 |

### REFERENCES

Cheong, S., J. M. Clomburg, and R. Gonzalez, Energy- and carbon-efficient synthesis of functionalized small molecules in bacteria using non-decarboxylative Claisen condensation reactions. Nature Biotechnology, 2016. 34(5): p. 556-561.
Dellomonaco, C., et al., Engineered reversal of the beta-oxidation cycle for the synthesis of fuels and chemicals. Nature, 2011. 476(7360): p. 355-U131.
Dudley, Q. M., C. J. Nash, and M. C. Jewett, Cell-free biosynthesis of limonene using enzyme enriched Escherichia coli lysates. Synthetic Biology, 2019. 4(1).
Karim, A. S., et al., In vitro prototyping and rapid optimization of biosynthetic enzymes for cell design. Nature Chemical Biology, 2020.
Luo, X. Z., et al., Complete biosynthesis of cannabinoids and their unnatural analogues in yeast. Nature, 2019. 567(7746): p. 123-+.
Omura, S., Antibiotic Cerulenin, a Novel Tool for Biochemistry as an Inhibitor of Fatty-Acid Synthesis. Bacteriological Reviews, 1976. 40(3): p. 681-697.
Tan, Z. G., J. M. Clomburg, and R. Gonzalez, Synthetic Pathway for the Production of Olivetolic Acid in Escherichia coli. Acs Synthetic Biology, 2018. 7(8): p. 1886-1896.
Valliere, M. A., et al., A cell-free platform for the prenylation of natural products and application to cannabinoid production. Nature Communications, 2019. 10.

### ADDITIONAL REFERENCES

Patent documents: US20160362708A1; WO2020028722A1; WO2008039499A3; US20160010126A1; US20160340629A1; WO2017139496A1; WO2019123152; US20190085347A1; US20190271010A1; EP3325634A4; U.S. Pat. No. 8,357,529B2; WO2016168708A1; US20190100741A1; U.S. Pat. No. 9,416,364B2.
Clomburg et al. A Synthetic Biology Approach to Engineer a Functional Reversal of the β-Oxidation Cycle. ACS Synth Biol, 1, 11, 541-554 (2012).
Clomburg, J M. et al. Integrated engineering of β-oxidation reversal and ω-oxidation pathways for the synthesis of medium chain ω-functionalized carboxylic acids. Metab Eng. 202-212 (2015).
Dudley et al. Cell-free prototyping of limonene biosynthesis using cell-free protein synthesis. bioRxiv (2020).
Haapalainen, A. M., et al., The thiolase superfamily: condensing enzymes with diverse reaction specificities. Trends Biochem. Sci. 31, 64-71 (2006).
Karim, A. S. & Jewett, M. C. A cell-free framework for rapid biosynthetic pathway prototyping and enzyme discovery. Metabolic Engineering 36, 116-126 (2016).
Price et al. Inhibition of β-Ketoacyl-Acyl Carrier Protein Synthases by Thiolactomycin and Cerulenin. JBC 276, 6551-6559 (2001).
Stehle et al. Designing microorganisms for heterologous biosynthesis of cannabinoids. FEMS Yeast Res 17 (2017).
Teixeira, P G. et al. Quantitative in vivo evaluation of the reverse β-oxidation pathway for fatty acid production in Saccharomyces cerevisiae. bioRxiv (2016).

Citations to a number of patent and non-patent references are made herein. The cited references are incorporated by reference herein in their entireties. In the event that there is an inconsistency between a definition of a term in the specification as compared to a definition of the term in a cited reference, the term should be interpreted based on the definition in the specification.

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

All methods described herein can be performed in any suitable order unless otherwise indicated herein or otherwise clearly contradicted by context. The use of any and all examples provided herein, is intended merely to better illuminate the invention and does not pose a limitation on the scope of the invention unless otherwise claimed. No language in the specification should be construed as indicating any non-claimed element as essential to the practice of the invention.

It will be understood by one of ordinary skill in the art that reaction components are routinely stored as separate solutions, each containing a subset of the total components, for reasons of convenience, storage stability, or to allow for application-dependent adjustment of the component concentrations, and that reaction components are combined prior to the reaction to create a complete reaction mixture. Furthermore, it will be understood by one of ordinary skill in the art that reaction components are packaged separately for commercialization and that useful commercial kits may contain any subset of the reaction components of the invention.

The methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

We claim:

1. A method for the enzymatic preparation of hexanoic acid or an intermediate of hexanoic acid in a hexanoic acid synthetic pathway in vitro from a feedstock comprising glucose or a product of glycolysis that reacts with one or more enzymes to produce the hexanoic acid or the intermediate of hexanoic acid in the hexanoic acid synthetic pathway, the method comprising:
(a) reacting a cell-free protein synthesis reaction mixture, the cell-free protein synthesis reaction mixture comprising a cellular extract from a host strain, a translation template encoding the one or more enzymes, and cell-free protein synthesis reagents,
(b) expressing the translation template in the cell-free protein synthesis reaction mixture to prepare the one or more enzymes,
(c) combining the cell-free protein synthesis reaction mixture and the feedstock to form a secondary reaction mixture,
wherein the feedstock reacts in the presence of the one or more enzymes to produce the hexanoic acid or the intermediate of hexanoic acid in the hexanoic acid synthetic pathway;
wherein the one or more enzymes are selected from the group consisting of acetyl-CoA acetyltransferase (ThlA), β-hydroxybutyryl-CoA dehydrogenase (Hbd1), 3-hydroxybutyryl-CoA dehydratase (Crt), trans-enoyl-CoA reductase (Ter), thioesterase 1 (TesA), and combinations thereof, and
wherein the intermediate of hexanoic acid in the hexanoic acid synthetic pathway is selected from acetyl-CoA, acetoacetyl-CoA, 3-hydroxybutyryl-CoA, hex-(2E)-enoyl-CoA, and hexanoyl-CoA.

2. A method for the enzymatic preparation of olivetolic acid or an intermediate of olivetolic acid in a olivetolic acid synthetic pathway in vitro from a feedstock comprising glucose or a product of glycolysis and hexanoyl-CoA that reacts with one or more enzymes to produce the olivetolic acid or the intermediate of olivetolic acid in the olivetolic acid synthetic pathway, the method comprising:
(a) reacting a cell-free protein synthesis reaction mixture, the cell-free protein synthesis reaction mixture comprising a cellular extract from a host strain, a translation template encoding the one or more enzymes, and cell-free protein synthesis reagents,
(b) expressing the translation template in the cell-free protein synthesis reaction mixture to prepare the one or more enzymes,
(c) combining the cell-free protein synthesis reaction mixture and the feedstock to form a secondary reaction mixture,
wherein the feedstock reacts in the presence of the one or more enzymes to produce the olivetolic acid or the intermediate of olivetolic acid in the olivetolic acid synthetic pathway;
wherein the one or more enzymes are selected from the group consisting of acetyl coenzyme A carboxylase (AccBCDA) and biotin-[acetyl-CoA-carboxylase]ligase (BirA), 3,5,7-Trioxododecanoyl-CoA synthase (TKS), olivetolic acid cyclase (OAC), and combinations thereof, and
wherein the intermediate of olivetolic acid in the olivetolic acid synthetic pathway is selected from acetyl-CoA, malonyl-CoA, hexanoyl-CoA, and 3,5,7-trioxododecanoyl-CoA.

3. A method for the enzymatic preparation of cannabigerolic acid or an intermediate of cannabigerolic acid in a cannabigerolic acid synthetic pathway in vitro from a feedstock comprising glucose or a product of glycolysis and olivetolic acid that reacts with one or more enzymes to produce the cannabigerolic acid or the intermediate of cannabigerolic acid in the cannabigerolic acid synthetic pathway, the method comprising:
(a) reacting a cell-free protein synthesis reaction mixture, the cell-free protein synthesis reaction mixture comprising a cellular extract from a host strain, a translation template encoding the one or more enzymes, and cell-free protein synthesis reagents,
(b) expressing the translation template in the cell-free protein synthesis reaction mixture to prepare the one or more enzymes,
(c) combining the cell-free protein synthesis reaction mixture comprising the feedstock to form a secondary reaction mixture,
wherein the feedstock reacts in the presence of the one or more enzymes to produce the cannabigerolic acid or the intermediate of cannabigerolic acid in the cannabigerolic acid synthetic pathway;
wherein the one or more enzymes are selected from the group consisting of acetyl-CoA acetyltransferase (AtoB), 3-hydroxy-3-methyl-glutaryl-CoA synthase (HMGS), 3-hydroxy-3-methyl-glutaryl-coenzyme A reductase (HMGR), mevalonate kinase (Mk), phosphomevalonate kinase (PMK), pyrophosphonevalonate decarboxylase (PMD), isopentenyl pyrophosphate isomerase (IDI), geranyl diphosphate synthase (GPPS), and prenyltransferase NphB7, and combinations thereof, and
wherein the intermediate of cannabigerolic acid in the cannabigerolic acid synthetic pathway is selected from acetyl-CoA, mevalonate, and geranyl pyrophosphate.

4. The method of claim 1, comprising adding NAD+ and coenzyme A to the secondary reaction mixture.

5. The method of claim 2, comprising adding cerulenin to the secondary reaction mixture.

6. The method of claim 1, wherein the host strain for the cellular extract comprises Escherichia coli (E. coli).

7. The method of claim 6, wherein the host strain comprises one or more of E. coli strain BL21, JS07, MB263, MB263sucD and JC01.

8. The method of claim 7, wherein the host strain comprises JS07.

9. The method of claim 1, wherein the cell-free protein synthesis reaction mixture and the secondary reaction mixture are in separate reaction vessels.

10. The method of claim 1, wherein the cell-free protein synthesis reaction and the secondary reaction are in the same reaction vessel.

11. A kit comprising:
a) a first composition comprising acetyl-CoA acetyltransferase (ThlA), (3-hydroxybutyryl-CoA dehydrogenase (Hbd1), 3-hydroxybutyryl-CoA dehydratase (Crt), trans-enoyl-CoA reductase (Ter), thioesterase 1 (TesA);
b) a second composition comprising acetyl coenzyme A carboxylase (AccBCDA) and biotin-[acetyl-CoA-carboxylase]ligase (BirA), 3,5,7-Trioxododecanoyl-CoA synthase (TKS), olivetolic acid cyclase (OAC); and
c) a third composition comprising acetyl-CoA acetyltransferase (AtoB), 3-hydroxy-3-methyl-glutaryl-CoA synthase (HMGS), 3-hydroxy-3-methyl-glutaryl-coenzyme A reductase (HMGR), mevalonate kinase (Mk), phosphomevalonate kinase (PMK), pyrophosphomevalonate decarboxylase (PMD), isopentenyl pyrophosphate isomerase (IDI), geranyl diphosphate synthase (GPPS), and prenyltransferase NphB7.

12. The kit of claim 11, wherein the second composition comprises cerulenin.

13. The kit of claim 11, wherein at least one of the first, second, and third composition comprises a cell extract.

14. The kit of claim 13, wherein the cell extract comprises an E. coli cell extract.

15. The kit of claim 14, wherein the E. coli extract comprises a JS07 extract.

16. The method of claim 2, wherein the hexanol-CoA is prepared by the method of claim 1.

17. The method of claim 3, wherein the olivetolic acid is prepared by the method of claim 2.

18. The method of claim 2, wherein the host strain for the cellular extract comprises Escherichia coli (E. coli).

19. The method of claim 3, wherein the host strain for the cellular extract comprises Escherichia coli (E. coli).

20. The method of claim 2, wherein the cell-free protein synthesis reaction and the secondary reaction are in the same reaction vessel.
